(19) United States
(12) Patent Application Publication
(10) Pub. No.: US 2008/0226740 A1
(43) Pub. Date: Sep. 18, 2008

(54) Marine algal extracts comprising marine algal polysaccharides of low degree polymerizaton, and the preparation processes and uses thereof

(75) Inventors: Rong-Huei Chen, Taipei (TW); Szu-Kai Chen, Taipei (TW); Wei-Yu Chen, Yun-Lin Hsien (TW); Szu-Hui Chen, Taichung Hsien (TW); Kun-Chi Huang, Kaohsiung Hsien (TW); Chia-Sui Hsu, Taichung Hsien (TW); Yo-Ru Hsu, Taichung (TW); Shi-Wei Bai, Chung-Li City (TW)

Correspondence Address: BIRCH STEWART KOLASCH & BIRCH, PO BOX 747, FALLS CHURCH, VA, 22040-0747, US

(73) Assignee: NATIONAL TAIWAN OCEAN UNIVERSITY, Keelung (TW)

(21) Appl. No.: 11/898,498

(22) Filed: Sep. 12, 2007

(30) Foreign Application Priority Data
Mar. 14, 2007 (TW) 096108863

(51) Int. Cl. A61K 36/02; A61K 9/14

(52) U.S. Cl. 424/499; 424/195.17

(57) ABSTRACT

Disclosed herein are marine algal extracts containing marine algal polysaccharides of low degree polymerization, and nanoparticles fabricated from the extracts. Preparation processes and applications of the marine algal extracts and the nanoparticles are also disclosed.

### CROSS-REFERENCE TO RELATED APPLICATION

This application claims priority of Taiwan Application No. 096108863, filed on Mar. 14, 2007.

### BACKGROUND OF THE INVENTION

1. Field of the Invention

This invention relates to a marine algal extract comprising marine algal polysaccharides of low degree polymerization, and the preparation process and applications thereof. This invention also relates to a nanoparticle fabricated from said extract, as well as the preparation process and applications of the same.

2. Description of the Related Art

It has been known for a long time that the extracts of marine algae can be used as Agar-agar raw material. Marine algae that have been known for the production of Agar-agar raw material include, e.g., marine algae of Gelidium genus and Pterocladia genus of the family Gelidiaceae, and marine algae of Gracilaria genus of the family Gracilariaceae.

Marine algae of Gracilaria genus that appear in the sea area surrounding Taiwan include: Gracilaria arcuata, Gracilaria blodgettii, Gracilaria bursa-pastoris, Gracilaria canaliculata, Gracilaria chorda, Gracilaria coronopifolia, Gracilaria edulis, Gracilaria eucheumoides, Gracilaria gigas, Gracilaria gracilis, Gracilaria incurvata, Gracilaria punctata, Gracilaria salicomia, Gracilaria spinulosa, Gracilaria srilankia, Gracilaria textorii, and Gracilaria veillardii (see the Taiwan Biodiversity National Information Network at the website of http://taibnet.sinica.edu.tw/home.asp). At present, there are five marine algae of Gracilaria genus that are cultivated in the sea area surrounding Taiwan, including Gacilaria coforvoides, Gracilaria gigas, Gracilaria chorda, Gracilaria lichenoides, and Gracilaria compressa, with the former two being particularly suitable for algal cultivation in aquaculture ponds.

Marine algae of Gelidium genus that appear in the sea area surrounding Taiwan include: Gelidium amansii, Gelidium corneum, Gelidium crinale, Gelidium divaricatum, Gelidium elegans Kutzing, Gelidium foliaceum, Gelidium japonicum, Gelidium kintaroi, Gelidium latiusculum, Gelidium pacificum, Gelidium planiusculum, Gelidium pusillim, Gelidium pusillum, Gelidium yamadae, etc. (see the Taiwan Biodiversity National Information Network at the website of http://taibnet.sinica.edu.tw/home.asp).

Marine algae of Pterocladia genus of the family Gelidiaceae that appear in the sea area surrounding Taiwan include: Pterocladia tenuis, Pterocladia nana, and Pterocladiella capillacea (see the Taiwan Biodiversity National Information Network at the website of http://taibnet.sinica.edu.tw/home.asp).

Agar-agar has a variety of industrial applications due to the gelling property, viscosity and emulsifying property thereof. For example, it can be used in the industrial fields as shown in the following Table 1.

TABLE 1

| | |
|---|---|
| Food industry | For the manufacture of confections, beverages, flavoring |
| | agents, dairy products, canned goods, etc. |
| Biochemical analyses | For the isolation and purification of biological substances such |
| | as microbial toxins, antibiotics, enzymes and the like, for |
| | detecting the size of virus particles, for the formulation of cell |
| | culture media, etc. |
| Chemical industry | For enhancing the foaming power of detergents, for use in |
| | corrosion prevention of iron/aluminum, for the manufacture of |
| | insecticides, water repellents, water paints, rubber stock |
| | solutions, foaming agents, lubricants, film-forming agents, |
| | printing primers, insect-proof papers, sheet papers, etc. |
| Pharmaceutical industry | For the fabrication of dental teeth models, dental adhesives |
| | and sealants, medicines, etc. |
| Cosmetics | For the manufacture of lotions, face creams, tooth pastes, |
| | essential oils, hair creams, etc. |
| Architecture Industry | For the prevention of sedimentation, for the production of deep |
| | well cement, etc. |
| Others | For wine brewing, for the production of crepe thickeners, etc. |

In addition, it has reported that agar-agar can serve as a health food for lowering blood lipid and cholesterol.

Most of the prior processes used hot water, acids, bases or enzymes to extract agar-agar from marine algae. There has been an early report indicating that agar-agar is comprised of neutral agarose and charged agaropectin (M. Duckworth and W. Yaphe. (1971), Carbohydrate Research, 16:189-197). However, the statement of this report in fact is too simple.

A further report indicates that agar-agar comprises three structural components having a common backbone structure. The first structural component is neutral agarose, which is a disaccharide polymer having a molecular weight of from 100,000 to 120,000 Daltons and composed of 1,3-substituted β-D-galactopyranosyl (unit A) and 1,4-substituted 3-6-anhydro-α-L-galactopyranosyl (unit B), in which none of unit A and unit B is charged. The neutral agarose may contain therein 6-O-methyl-D-galactose. The second structural component has a backbone structure similar to that of the first structural component, but that unit A is replaced by pyruvic acid acetal and the proportion of pyruvic acid to D-galactose is approximately 1:51, and a low degree of sulphation is present within the second structural component. The third structural component is sulphated galactan, which contains very low or even no unit B and pyruvic acid. It is a non-gelling galactan due to the high degree of sulphation thereof (L. G. Enriquez and G. J. Flick. (1989), Food emulsifiers: chemistry, technology functional properties and applications. pp. 235-334).

In view of the fact that marine algae are universally available, currently researches aimed at marine algal extracts have been rapidly developed, in particular regarding the applications thereof in the treatment of diseases and in the manufacture of pharmaceutics. For example, KR 2004057103 A discloses a therapeutic composition for the treatment of degenerative joint disease, the composition being characterized by a marine algal extract containing 10% or more polyphloroglucinol complex (PPC). Referring to the Specification thereof, marine algae suitable for use in the composition of KR 2004057103 A do not include those of Gracilaria genus.

KR 2005034904 A discloses an extract of Gelidium amansii capable of activating gut immunity and the preparation process thereof, which involves a step of using ethanol to extract a dry material of Gelidium amansii.

KR 2004057021 A discloses a composition for inhibiting the differentiation of NIH3T3-L1 cells, comprising an active fraction obtained from Gelidium amansii. The composition can also lower the blood glucose level and, hence, can be used in the prevention or treatment of diabetes.

JP 45018646 B discloses the use of H₂NSO₃H or CISO₃H to treat agar or non-extracted agar. In addition, JP 47020007B discloses the use of H₂O₂ to decompose agar or Gelidium amansii, followed by sulfation. The product disclosed therein can be used as an antiulcer agent, an antipepsin and an anti-inflammatory agent.

Additionally, there have been a series of Japanese patent publications that relate to the extracts of Gracilaria sp. and the preparation process and applications of the same. JP 2006104117A, JP 2006104118A and JP 2006104180A disclose the use of an aqueous salt solution to extract Gracilaria sp., adding ammonium sulfate to the obtained extract solution up to a final concentration of 20 to 40% saturated concentration to perform a first salting-out treatment, followed by removal of precipitated impurities. Thereafter, ammonium sulfate was further added to the obtained extract solution up to a final concentration of 60 to 80% saturated concentration to perform a second salting-out treatment, followed by recovery of a crude active fraction as precipitates. Finally, the precipitates were dissolved in a proper solvent to separate and collect the liquid extract exhibiting a cell-mediated immunity potentiating activity. The extracts of Gracilaria sp. exhibited a high mitogen activity, promising the use thereof in the preparation of skin anti-aging compositions, skin-lightening compositions and the like, and exhibited physiological activities, such as the activity of activating cellular immunocompetence, thus being useful in restoring photo-inhibited immunocompetence.

EP 295956 A2 discloses polysaccharides extracted from marine algae could be used in the treatment of viral infections, such as the treatment of AIDS. According to EP 295956 A2, the polysaccharides were obtained by extracting a marine alga with an aqueous solvent, followed by refining the resultant extract.

There is another report disclosing the extraction of 27 species of common seaweeds by using organic solvent(s), so as to obtain extracts having anti-oxidative activity. According to this report, seaweeds were pulverized into powder and sequentially extracted by chloroform, ethyl acetate, acetone and methanol to give four different organic extracts. The residue left after organic solvent extraction was extracted with water to give a water phase extract. Thereafter, free radical scavenging activity and hydroxyl radical scavenging activity were measured by 1,1-diphenyl-2-picrylhydrazyl assay (DPPH assay) and deoxyribose assay, respectively (Yan et al. (1998), Plant Foods for Human Nutrition, 52:253-262).

In contrast to the extraction treatments using (NH₄)₂SO₄ or organic solvent(s), Chen et al. reported that dry powdered Gelidium amansii was extracted with phosphate-buffered saline (PBS) to give an aqueous solution. After centrifugation and filtration, the resultant supernatant was designated the PBS or water-soluble extract, and the remaining pellets were extracted with methanol to give a methanol extract. In addition, dry powdered Gelidium amansii was extracted by boiled water, followed by filtering, cooling and ultrasonication. Thereafter, the Gelidium amansii agar was freeze-dried to obtain Gelidium amansii agar powder, which was dissolved in DMSO prior to use in the experiments. Extracts of Gelidium amansii from various preparations exhibited anti-proliferative effects on Hepa-1 and NIH-3T3 cells, and apoptosis might play a role in the methanol and DMSO extract-induced inhibitory effects (Yue-Hwa Chen et al. (2004), Biol. Pharm. Bull., 27:180-184).

In a 2003 master thesis, entitled “Studies of the physiological activities of Gracilaria polysaccharides and their hydrolysates,” by Ma Ying-Yu, Department of Food Science, National Pingtung University of Science and Technology, it was reported that dry Gracilaria tenuistipitata was added into 2% NaOH, followed by a heating treatment in a 95° C. water bath. After washing with running water, an alkaline-treated alga was obtained. The alkaline-treated alga was immersed in a 0.2% acetic acid solution, added with distilled water and boiled, so as to reach a pH value up to 5.2. The resultant hot solution was filtered through gauze and allowed to cool at room temperature to give a gel-like product containing Gracilaria polysaccharides. The product was then lyophilized to form powder. Gracilaria polysaccharides were subjected to hydrolysis by enzymes (agarase and cellulase) or acids (hydrochloric acid and formic acid) or both acid and enzyme. The hydrolyzed products thus obtained were subjected to evaluation in terms of anti-oxidative activity, probiotic property and anti-hypercholesterolemia effect, respectively.

In the applicants' previous study, the applicants investigated the production of seaweeds mud mask and seaweeds mud bath and shower gel, the two products being produced by using a hot-water extract of Gracilaria lemaneformis to replace the corresponding ingredient(s) contained in the original formulation.

The basic formulation of the seaweeds mud mask is shown in the following Table 2, except that a hot-water extract of Gracilaria lemaneformis in powder form was used to replace the thickening agent, and kaolin or bentonite was used to replace the film forming agent. This product was prepared as follows: the buffering agent and the humectant were dissolved in pure water, followed by heating to 70˜80° C. After addition of the thickening agent and the film forming agent, the resultant mixture was evenly stirred to obtain an aqueous phase. Meanwhile, the preservative and the surfactant were dissolved in ethanol to form an oil phase. The oil phase was then poured into the aqueous phase with stirring. After cooling, the desired product was obtained.

TABLE 2

| Ingredients | Reagents | Percentage (%) |
|---|---|---|
| Film forming agent | Polyvinyl alcohol | 15.0 |
| Thickening agent | Methylcellulose | 2.0 |
| Humectant | 1,3-butandiol | 5.0 |
| Alcohol | Ethanol | 12.0 |
| Preservative | Methyl Paraben | 0.4 |
| Buffering agent | Sodium citrate | In an appropriate amount |
| Surfactant | POE oleyl alcohol ether | 0.5 |
| Pure water | | 65.1 |

Note:
The above formulation was based on the descriptions set forth on pages 13-46 of New Cosmetic Science (1992), edited by Takeo Mitsui, translated by Wei-Da Chen and Hue-Wen Cheng, pressed by Nanzando Co. Ltd., apan, published by Ho-Chi Book Publishing Co., Taiwan.

The basic formulation of the seaweeds mud bath and shower gel is shown in the following Table 3, except that mud of Gracilaria lemaneformis was used to replace the thickening agent. This product was prepared as follows: the de-ionized water in item A was heated to 45-55° C. while preventing bubble generation (using a vacuum heating stirrer if available). After dispersing Item B into item A with slow stirring, item C was added and the resultant mixture was slowly stirred. Disodium laureth sulphosuccinate was then added with slow stirring, followed by heating to 60-65° C. Cocamide MEA was added and the resultant mixture was heated to 65-67° C. until full dissolution was reached. The remaining ingredients of item D were added with slow stirring, and the resultant mixture was allowed to cool to 50° C. After the addition of item E, the resultant mixture was allowed to cool to 45° C. Item F was then added with slow stirring so as to prevent bubble generation. Thereafter, item G and item H were added in sequence with slow stirring until the fragrance dissolved. A product with evenly dispersed beads was then obtained.

TABLE 3

| Ingredients | Reagents | Percentage (%) |
|---|---|---|
| A | De-ionized water | 56.03 |
| A | Axrylates/C10-30 alkyl acrylate | 1.10 |
| | crosspolymer | |
| B | Triethanolamine | 0.1 |
| C | Tetrasodium EDTA(40% aq) | 0.12 |
| D | Disodium laureth sulphosuccinate | 13.0 |
| D | Cocamide MEA | 2.0 |
| D | Cocamidopropyl betaine | 5.00 |
| D | Sodium laureth sulphate | 15.00 |
| E | Diazolidinyl urea and iodopropylnyl | 0.2 |
| | butylcarbamate | |
| F | Triethanolamine(99%) | 1.45 |
| G | Fragrance | 1.00 |
| H | Hydrogenated jojoba oil | 5.0 |

Despite of the studies described above, it is the goal of relevant researchers to develop new products from marine algae that have great industrial values.

On the other hand, when used, agar-agar extracts face with the challenges of stability, absorptivity and gastric acid resistance. The development of nanofabrication processes happens to provide a new solution to deal with the above challenges. In particular, due to the controlled release effect thereof, the nanofabricated products have an increased targeting ability, and the active ingredients encapsulated within the nanoparticles can be well protected until they smoothly reach the target site for release. Besides, the enlarged surface areas of nanoparticles may contribute to the increase of absorptivity.

There have been abundant literatures on nanomaterials prepared from biological polymers. For example, as chitin and chitosan are excellent biomedical materials, in recent years, there have been many reports aiming at the development of nanomaterials using chitin or chitosan, alone or in combination with other biological polymers or synthetic polymers. The fabrication processes of nanomaterials include emulsion cross-linking, coacervation/precipitation, spray-drying, emulsion-droplet coacervation methods, ionic gelation, reverse micellar methods, and sieveing (Sunil A. Agnihotri et al., (2004), Journal of controlled release, 100:5-28).

KR 2004099189 A discloses a pharmacologically active microparticle using the extracts of 6 kinds of seaweeds including brown seaweed, sea tangle, Enteromorpha, layer, seaweed fusiforme and Gelidium amansii, in which the seaweeds were repeatedly subjected to extraction, concentration and filtration to give an extract. A ceramic material emitting energy was then dipped into the extract to give an active solution, which was passed through a magnetism treating apparatus to convert it into a microparticle.

An article further reported that insulin was admixed with a tripolyphosphate solution to form a mixture. After adding the mixture into a chitosan solution with even stirring, nanomaterials having a particle size ranging from 300-400 nm could be obtained when the concentration of the chitosan solution relative to that of the tripolyphosphate solution was 6:1 (R. Fernandez-Urrusuno et al. (1999), Pharm. Res., 16:1576-1581).

Despite of the studies described above, it is the goal of relevant investigators to develop new and useful nanoparticles.

### SUMMARY OF THE INVENTION

Therefore, according to a first aspect, this invention provides a marine algal extract that is produced by a process comprising the steps of:
(a) extracting a marine algal material with water at an elevated temperature, followed by removal of water insoluble substances, so that an aqueous extract containing marine algal polysaccharides is obtained;
(b) admixing the aqueous extract obtained from step (a) with an acid or an aqueous solution containing said acid so as to form an acidic aqueous solution;
(c) subjecting the acidic aqueous solution thus formed from step (b) to a refining treatment selected from heating treatments and ultrasonication treatments, so that a product containing acid-hydrolyzed marine algal polysaccharides is obtained; and
(d) subjecting the product obtained from step (c) to a ultrafiltration treatment having a molecular weight cut-off value ranging from 1×10² to 5×10⁴ Daltons, so that a marine algal extract comprising marine algal polysaccharides of low degree polymerization is obtained.

In a second aspect, this invention provides a process for producing a marine algal extract, comprising the steps of:
(a) extracting a marine algal material with water at an elevated temperature, followed by removal of water insoluble substances, so that an aqueous extract containing marine algal polysaccharides is obtained;
(b) admixing the aqueous extract obtained from step (a) with an acid or an aqueous solution containing said acid so as to form an acidic aqueous solution;
(c) subjecting the acidic aqueous solution thus formed from step (b) to a refining treatment selected from heating treatments and ultrasonication treatments, so that a product containing acid-hydrolyzed marine algal polysaccharides is obtained; and
(d) subjecting the product obtained from step (c) to a ultrafiltration treatment having a molecular weight cut-off value ranging from 1×10² to 5×10⁴ Daltons, so that a marine algal extract comprising marine algal polysaccharides of low degree polymerization is obtained.

In a third aspect, this invention provides a nanoparticle of chitosan-marine algal polysaccharides of low degree polymerization, said nanoparticle being produced by a process comprising the steps of:
(a) providing a reaction mixture by admixing a first aqueous solution containing chitosan and an acid with a second aqueous solution containing a marine algal extract as claimed in Claim 1; and
(b) subjecting the reaction mixture to a ultrasonication treatment, so that a third aqueous solution containing the nanoparticle is obtained.

In a fourth aspect, this invention provides a process for producing a nanoparticle of chitosan-marine algal polysaccharides of low degree polymerization, said process comprising the steps of:
(a) providing a reaction mixture by admixing a first aqueous solution containing chitosan and an acid with a second aqueous solution containing a marine algal extract as claimed in Claim 1; and
(b) subjecting the reaction mixture to a ultrasonication treatment, so that a third aqueous solution containing the nanoparticle is obtained.

The marine algal extract or the nanoparticle of chitosan-marine algal polysaccharides of low degree polymerization according to this invention have been demonstrated to have the effect of inhibiting melanin production by human melanoma cells, the effect of promoting fibroblast proliferation and/or collagen synthesis, and the effect of scavenging α,α-diphenyl-β-picryhydrazyl (DPPH) and superoxide radicals.

Therefore, in a fifth aspect, this invention provides a pharmaceutical composition or cosmetic product, which comprises an effective amount of the marine algal extract or the nanoparticle of chitosan-marine algal polysaccharides of low degree polymerization as described above. The pharmaceutical composition or cosmetic product according to this invention has the effect of inhibiting the growth of tumor cells (in particular the human melanoma cells) and the effect of promoting fibroblast proliferation and/or collagen synthesis.

### BRIEF DESCRIPTION OF THE DRAWINGS

The above and other objects, features and advantages of the present invention will become apparent with reference to the following detailed description and the preferred embodiments taken in conjunction with the accompanying drawings, in which:

FIG. 1 shows the influence of the reaction time of acetic acid hydrolysis upon the concentrations of galactose and total sugar of a hot-water extract of Gracilaria lemaneformis as prepared in Example 1, infra;

FIG. 2 shows the influence of the reaction time of acetic acid hydrolysis upon the average degree of polymerization of marine algal polysaccharides contained in the hot-water extract of Gracilaria lemaneformis as prepared in Example 1, infra, in which the results are represented by mean ±S.D. (n=4);

FIG. 3 shows the influence of the reaction time of acetic acid hydrolysis upon the specific viscosity of the hot-water extract of Gracilaria lemaneformis as prepared in Example 1, infra, in which the results are represented by mean ±S.D. (n=3);

FIG. 4 shows the effect of a marine algal extract of Gracilaria lemaneformis according to this invention (see Example 2, infra) in inhibiting melanin production by human melanoma cells A375, in which various concentrations (0.78˜400 μg/mL) of the extract were tested;

FIG. 5 shows the effect of a marine algal extract of Gracilaria lemaneformis according to this invention (designated with the abbreviation LDPGP, see Example 2, infra) in scavenging DPPH radicals, in which various concentrations (0.2%, 0.4%, 0.6%, 0.8% and 1.0% by weight) of the extract were tested, and a 1.0% BHT solution was used as a positive control group;

FIG. 6 shows the effect of a marine algal extract of Gracilaria lemaneformis according to this invention (designated with the abbreviation LDPGP, see Example 2, infra) in scavenging superoxide radicals, in which various concentrations (0.2%, 0.4%, 0.6%, 0.8% and 1.0% by weight) of the extract were tested and a 1.0% vitamin C solution was used as a positive control group;

FIG. 7 shows the reducing power of a marine algal extract of Gracilaria lemaneformis according to this invention (designated with the abbreviation LDPGP, see Example 2, infra) versus that of vitamin C, in which various concentrations of the extract were tested, and a 1.0% vitamin C solution was used as a control group;

FIG. 8 shows the effect of a marine algal extract of Gracilaria lemaneformis according to this invention (see Example 2, infra) upon the cell proliferation of human skin fibroblast cell line CCD-966SK, in which the cell proliferation rate was determined by the MTT method;

FIG. 9 shows the effect of a marine algal extract of Gracilaria lemaneformis according to this invention (designated with the abbreviation LDPGP, see Example 2, infra) upon the collagen synthesis by human skin fibroblast cell line CCD-966SK, in which various concentrations (1.5625, 3.75, 7.8, 15.625, 31.25, 62.5, 125 and 250 μg/mL) of the extract were tested, and the basal medium for cell culture was used as a control group;

FIG. 10 shows the effect of a vital cream having the basic formulation as shown in Table 4, infra, and containing a marine algal extract of Gracilaria lemaneformis according to this invention (designated with the abbreviation LDPGP, see Example 2, infra) upon the skin elasticity, in which the vital cream was applied to a skin area located at an inner side of each volunteer's lower arm for 3 weeks, and a cream of the same formulation but not containing the extract was used as a control group;

FIG. 11 shows the average particle size of nanoparticles that were prepared from a marine algal extract of Gracilaria lemaneformis as obtained from Example 2, infra, and chitosan of various concentrations (0.001, 0.01, 0.1 and 1% by weight), dissolved in a 0.05% acetic acid solution);

FIG. 12 shows the variation of average particle size of nanoparticles of chitosan-marine algal polysaccharides of low degree polymerization after different times of storage (0, 1, 5, 10, 20 and 30 days) at room temperature, the nanoparticles being prepared from chitosan and a marine algal extract of Gracilaria lemaneformis as obtained from Example 2, infra, with different times of ultrasonication (1, 2, 3, 4 and 5 minutes);

FIG. 13 shows the variation of average particle size of nanoparticles of chitosan-marine algal polysaccharides of low degree polymerization after storage at different temperatures (4° C., 30° C., and 50° C.) for 30 days, the nanoparticles being prepared from chitosan and a marine algal extract of Gracilaria lemaneformis as obtained from Example 2, infra, with 4 minutes of ultrasonication;

FIG. 14 shows the average particle size and zeta potential of nanoparticles of chitosan-marine algal polysaccharides of low degree polymerization as detected after different times of storage (0, 1, 5, 10, 20 and 30 days) at 30° C.;

FIG. 15 shows the variation of particle morphology of nanoparticles of chitosan-marine algal polysaccharides of low degree polymerization according to this invention as observed by scanning electron microscopy (SEM), in which panel A is a picture showing the nanoparticles before lyophilization, and panel B is a picture showing the nanoparticles after lyophilization;

FIG. 16 shows the effect of nanoparticles of nanoparticles of chitosan-marine algal polysaccharides of low degree polymerization as prepared in Example 11, infra, upon the cell proliferation rate of human skin fibroblast cell line CCD-966SK, in which various concentrations (0.375˜200 μg/mL) of the nanoparticles were tested, and the cell proliferation rate was determined by the MTT method;

FIG. 17 shows the effect of nanoparticles of chitosan-marine algal polysaccharides of low degree polymerization as prepared in Example 11, infra, in scavenging DPPH radicals, in which various concentrations (0.2%, 0.4%, 0.6%, 0.8% and 1.0% by weight) of the nanoparticles were tested, and a 1.0% vitamin E solution was used as a positive control group;

FIG. 18 shows the effect of nanoparticles of chitosan-marine algal polysaccharides of low degree polymerization as prepared in Example 11 in scavenging superoxide radicals, in which various concentrations (0.2%, 0.4%, 0.6%, 0.8% and 1.0% by weight) of the extract were tested and a 1.0% vitamin C solution was used as a positive control group;

FIG. 19 shows the reducing power of nanoparticles of nanoparticles of chitosan-marine algal polysaccharides of low degree polymerization as prepared in Example 11 versus that of vitamin C, in which various concentrations of the nanoparticles were tested, and a 1.0% vitamin C solution was used as a control group; and

FIG. 20 shows the effect of a vital cream having the basic formulation as shown in Table 6, infra, and containing nanoparticles as prepared in Example 11 upon the skin elasticity, in which the vital cream was applied to a skin area located at an inner side of each volunteer's lower arm at 25±1° C. and RH 60±1% for 3 weeks, and a cream of the same formulation but not containing the nanoparticles was used as a control group. The results were represented by means ±S.D. (n=12) (statistical significance, P<0.05).

### DETAILED DESCRIPTION OF THE INVENTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art, in Taiwan or any other country.

Unless defined otherwise, all technical and scientific terms used herein have the meaning commonly understood by a person skilled in the art to which this invention belongs. One skilled in the art will recognize many methods and materials similar or equivalent to those described herein, which could be used in the practice of the present invention. Indeed, the present invention is in no way limited to the methods and materials described. For clarity, the following definitions are used herein.

This invention provides a marine algal extract produced by a process comprising the steps of:
(a) extracting a marine algal material with water at an elevated temperature, followed by removal of water insoluble substances, so that an aqueous extract containing marine algal polysaccharides is obtained;
(b) admixing the aqueous extract obtained from step (a) with an acid or an aqueous solution containing said acid so as to form an acidic aqueous solution;
(c) subjecting the acidic aqueous solution thus formed from step (b) to a refining treatment selected from heating treatments and ultrasonication treatments, so that a product containing acid-hydrolyzed marine algal polysaccharides is obtained; and
(d) subjecting the product obtained from step (c) to a ultrafiltration treatment having a molecular weight cut-off value ranging from 1×10² to 5×10⁴ Daltons, so that a marine algal extract comprising marine algal polysaccharides of low degree polymerization is obtained.

According to this invention, the marine algal material as used in step (a) of said process belongs to any of the following: a marine alga of Gracilaria genus, and a marine alga of the family Geidiaceae.

Preferably, the marine algal material as used in step (a) of said process belongs to any of the following: Gracilaria coforvoides, Gracilaria gigas, Gracilaria chorda, Gracilaria lichenoides, Gracilaria compressa, Gracilaria arcuata, Gracilaria blodgettii, Gracilaria bursa-pastoris, Gracilaria canaliculata, Gracilaria lemaneformis, Gracilaria coronopifolia, Gracilaria edulis, Gracilaria eucheumoides, Gracilaria gracilis, Gracilaria incurvata, Gracilaria punctata, Gracilaria salicornia, Gracilaria spinulosa, Gracilaria srilankia, Gracilaria textori, Gracilaria veillardii, Gelidium amansii, Gelidium corneum, Gelidium crinale, Gelidium divaricatum, Gelidium elegans, Gelidium foliaceum, Gelidium japonicum, Gelidium kintaroi, Gelidium latiusculum, Gelidium pacificum, Gelidium planiusculum, Gelidium pusillim, Gelidium pusillum, Gelidium yamadae, Pterocladia tenuis, Pterocladia nana, and Pterocladiella capillacea.

More preferably, the marine algal material as used in step (a) of said process belongs to any of the following: Gracilaria coforvoides, Gracilaria gigas, Gracilaria chorda, Gracilaria lemaneformis, Gracilaria lichenoides, and Gracilaria compressa.

In a preferred embodiment of this invention, the marine algal material as used in step (a) of said process belongs to Gracilaria lemaneformis.

According to this invention, the marine algal material to be used in step (a) of said process can be subjected to preliminary treatments including washing, drying and cutting/crushing. Alternatively, the marine algal material is in the form of a dried algal body and it can be cut into small pieces, washed with dd water, and immersed for 1-3 hrs, so as to remove impurities and soften the algal body.

According to this invention, step (a) of said process is conducted at a temperature ranging from 70° C. to 100° C. for a period of from 1 to 6 hours. In a preferred embodiment of this invention, the marine algal material is admixed with water and then extracted at 100° C. for 6 hrs.

According to this invention, in step (a) of said process, removal of water insoluble substances is conducted by filtration or centrifugation.

According to this invention, the aqueous extract as obtained from step (a) of said process is in the form of an aqueous solution and is admixed with said acid in step (b). Alternatively, the aqueous extract as obtained from step (a) of said process is in the form of a lyophilized powder and is admixed with an aqueous solution containing said acid in step (b).

According to this invention, the acid used in step (b) of said process is an organic acid or an inorganic acid. Preferably, the acid used in step (b) of said process is an inorganic acid selected from the group consisting of hydrochloric acid, nitric acid, phosphoric acid, and combinations thereof. Preferably, the acid used in step (b) of said process is an organic acid selected from the group consisting of acetic acid, formic acid, lactic acid, malic acid, oxalic acid, citric acid, and combinations thereof. In a preferred embodiment of this invention, the acid used in step (b) of said process is acetic acid.

According to this invention, said acid or the aqueous solution containing said acid to be used in step (b) of said process has a concentration in the range of from 0.01% to 30%. Preferably, said acid or the aqueous solution containing said acid to be used in step (b) of said process has a concentration in the range of from 0.01% to 15%. More preferably, said acid or the aqueous solution containing said acid to be used in step (b) of said process has a concentration in the range of from 0.01% to 10%. Most preferably, said acid or the aqueous solution containing said acid to be used in step (b) of said process is an aqueous acetic acid solution having a concentration in the range of from 0.01% to 10%.

According to this invention, in step (c) of said process, the acidic aqueous solution thus formed from step (b) is subjected to a heating treatment. Preferably, the heating treatment is conducted at a temperature ranging from 70° C. to 100° C. More preferably, the heating treatment is conducted at a temperature ranging from 80° C. to 95° C. More preferably, the heating treatment is conducted at a temperature ranging from 80° C. to 90° C. In a preferred embodiment of this invention, the heating treatment is conducted at a temperature of 90° C.

According to this invention, the heating treatment is conducted for a period of from 0.1 to 10 hours. Preferably, the heating treatment is conducted for a period of from 4 to 9 hours. More preferably, the heating treatment is conducted for a period of from 5 to 7 hours.

Alternatively, in step (c) of said process, the acidic aqueous solution thus formed from step (b) is subjected to a ultrasonication treatment. Preferably, the ultrasonication treatment is conducted at a temperature ranging from 70° C. to 100° C.

According to this invention, the ultrasonication treatment is conducted at a power of from 10 to 1000 watts.

As used herein, the term “degree of polymerization” refers to the number of repeating units contained in a polymeric molecule. Therefore, the term “marine algal polysaccharide of low degree polymerization” refers to a marine algal polysaccharide having a small molecular weight and is used interchangeably with the term “marine algal oligosaccharide.”

According to this invention, the marine algal extract thus obtained from said process comprises marine algal polysaccharides of low degree polymerization that have a molecular weight in the range of from 1×10² to 5×10⁴ Daltons. Preferably, the marine algal extract comprises marine algal polysaccharides of low degree polymerization that have a molecular weight in the range of from 1×10² to 1×10⁴ Daltons. More preferably, the marine algal extract comprises marine algal polysaccharides of low degree polymerization that have a molecular weight in the range of from 1×10² to 5×10³ Daltons.

On the other hand, in order to accomplish large scale production of nanoparticles comprising the marine algal extract according to this invention, the applicants surprisingly found that chitosan is a very suitable material to achieve this goal.

Accordingly, this invention also provides a nanoparticle of chitosan-marine algal polysaccharides of low degree polymerization, said nanoparticle being produced by a process comprising the steps of:
(a) providing a reaction mixture by admixing a first aqueous solution containing chitosan and an acid with a second aqueous solution containing a marine algal extract as claimed in Claim 1; and
(b) subjecting the reaction mixture to a ultrasonication treatment, so that a third aqueous solution containing the nanoparticle is obtained.

According to this invention, a nanoparticle having an appropriate particle size and zeta potential can be produced by adjusting the used amounts of the first and second aqueous solutions.

According to this invention, in step (a) of said process, the used amount of the first aqueous solution versus that of the second aqueous solution is within the range of from 1:1 to 10:1. In a preferred embodiment of this invention, the used amount of the first aqueous solution versus that of the second aqueous solution is 1:1. In another preferred embodiment of this invention, the used amount of the first aqueous solution versus that of the second aqueous solution is 2:1. In a further preferred embodiment of this invention, the used amount of the first aqueous solution versus that of the second aqueous solution is 3:1.

According to this invention, the first aqueous solution used in step (a) of said process has a concentration of chitosan in the range of from 0.002% to 1.0% by weight. Preferably, the first aqueous solution has a concentration of chitosan in the range of from 0.006% to 0.5% by weight. More preferably, the first aqueous solution has a concentration of chitosan in the range of from 0.01% to 0.2% by weight.

According to this invention, the second aqueous solution used in step (a) of said process has a concentration of marine algal polysaccharides of low degree polymerization in the range of from 0.0010% to 0.5% by weight. Preferably, the second aqueous solution has a concentration of marine algal polysaccharides of low degree polymerization in the range of from 0.001% to 0.2% by weight.

According to this invention, in step (a) of said process, the acid contained in the first aqueous solution is an organic acid or an inorganic acid. In a preferred embodiment of this invention, the acid is an inorganic acid selected from the group consisting of hydrochloric acid, nitric acid, phosphoric acid, and combinations thereof. In another preferred embodiment of this invention, the acid is an organic acid selected from the group consisting of acetic acid, formic acid, lactic acid, malic acid, oxalic acid, citric acid, and combinations thereof.

Preferably, the first aqueous solution used in step (a) of said process comprises an aqueous acetic acid solution having a concentration in the range of from 0.01% to 30%. More preferably, the first aqueous solution used in step (a) of said process comprises an aqueous acetic acid solution having a concentration in the range of from 0.01% to 10%. Most preferably, the first aqueous solution used in step (a) of said process comprises an aqueous acetic acid solution having a concentration in the range of from 0.01% to 1%.

The applicants also found that some factors might influence the particle size and storage stability of the nanoparticle obtained from the process according to this invention. These factors include: the temperature and time of the ultrasonication treatment, and the time and temperature of storage. Therefore, it is appreciable that one skilled in the art can readily determine the operating parameters of the process disclosed herein, including the temperature and time of the ultrasonication treatment, and the time and temperature of storage, so as to obtain highly stable nanoparticles of chitosan-marine algal polysaccharides of low degree polymerization.

According to this invention, in step (b) of said process, the ultrasonication treatment is conducted at a temperature ranging from 4° C. to 50° C. Preferably, the ultrasonication treatment is conducted at a temperature ranging from 10° C. to 40° C. More preferably, the ultrasonication treatment is conducted at a temperature ranging from 25° C. to 35° C. In a preferred embodiment of this invention, the ultrasonication treatment is conducted at 30° C.

According to this invention, in step (b) of said process, the ultrasonication treatment is conducted at a power of from 20 to 100 watts.

According to this invention, in step (b) of the process, the ultrasonication treatment is conducted for a period of from 1 to 60 minutes. Preferably, the ultrasonication treatment is conducted for a period of from 1 to 30 minutes. More preferably, the ultrasonication treatment is conducted for a period of from 1 to 10 minutes. In a preferred embodiment of this invention, the ultrasonication treatment is conducted for 4 minutes.

According to this invention, the third aqueous solution thus obtained from step (b) of said process can be further purified by the following step:
(c) subjecting the third aqueous solution thus obtained from step (b) to a high-speed centrifugation treatment, so that a supernatant containing the nanoparticle may be collected.

According to this invention, the high-speed centrifugation treatment is conducted at a speed ranging from 5000 to 20000 rpm. Preferably, the high-speed centrifugation treatment is conducted at a speed ranging from 8000 to 15000 rpm.

According to this invention, the nanoparticle contained in the supernatant thus obtained from step (c) can be recovered by methods well known in the art, including but not limited to lyophilization, spray-drying, evaporation, heat-drying, and a combination thereof.

The marine algal extract or the nanoparticle of chitosan-marine algal polysaccharides of low degree polymerization according to this invention have been demonstrated to have the effects of inhibiting melanin production by human melanoma cells, promoting fibroblast proliferation and/or collagen synthesis, and scavenging α,α-diphenyl-β-picryhydrazyl (DPPH) and superoxide radicals. Therefore, it is contemplated that the marine algal extract or the nanoparticle of chitosan-marine algal polysaccharides of low degree polymerization according to this invention have a great potential in the manufacture of anti-aging health products, pharmaceutical compositions for inhibiting the growth of tumor cells (in particular the human melanoma cells), pharmaceutical compositions or cosmetic products for inhibiting melanin synthesis or for promoting fibroblast proliferation and/or collagen synthesis, and dressings for wound healing.

The marine algal extract or the nanoparticle of chitosan-marine algal polysaccharides of low degree polymerization according to this invention can be employed in combination with any of the additive(s) that are commonly used in the art, e.g., hydrophilic or lipophilic gelling agents, hydrophilic or lipophilic active agents, preserving agents, antioxidants, solvents, fragrances, fillers, screening agents, colors, chelating agents, odor absorbers, and dyes. Each of the additives is used in an amount that is determined based on the ordinary practice in the art.

The marine algal extract or the nanoparticle of chitosan-marine algal polysaccharides of low degree polymerization according to this invention can be prepared in any dosage form, including, but not limited to: aqueous solution, sterile power, tablet, capsule, water-alcohol solution or oily solution, emulsions of either oil-in-water type, water-in-oil type or other multi-phase systems, aqueous or oily gel, cream, ointment, milk, lotion, serum, paste, foam, dispersion, etc.

The marine algal extract or the nanoparticle of chitosan-marine algal polysaccharides of low degree polymerization according to this invention can also be prepared as a cosmetic product of any sort and type, including, but not limited to, tonic waters, lip colors, foundations, milks, creams, masks, gels, aerosols, milky lotions, mousses, dispersions, creams, toilet waters, packs and cleansings, cleansers for make-up removal, wash soaps, etc.

In addition, the cosmetic products according to this invention can further comprise other whitening agents and other active ingredients known to be beneficial to whitening, including, but not limited to: tyrosinase inhibitors, such as vitamin C, arbutin, kojic acid, quercetin, catechin, etc., anti-acne agents, antibacterial agents, analgesics, anesthetics, anti-cutaneous inflammatory agents, antipruritics, anti-inflammatory agents, anti-hyperkeratolytic agents, anti-dry skin agents, anti-psoriatic agents, anti-aging agents, anti-wrinkle agents, anti-seborrheic agents, self-tanning agents, wound-healing agents, corticosteroids, hormones, etc.

The marine algal extract or the nanoparticle of chitosan-marine algal polysaccharides of low degree polymerization according to this invention can be prepared as a cosmetic product in combination with an external dermal agent. As used herein, the term “external dermal agent” refers to a reagent or ingredient that is commonly used in cosmetic products or medicaments for external use, including, but not limited to, other skin whitening agents, humectants, antioxidants, UV absorbants, surfactants, thickeners, colors, skin nutrients, etc.

Optionally, when preparing an oral preparation comprising the marine alga extract or the nanoparticles of chitosan-marine algae polysaccharides of low degree polymerization according to this invention, the oral preparation can further comprise an excipient and, if desired, a binder, a disintegrator, a lubricant, a coloring matter, a flavoring agent and/or the like. The oral preparation can then be formed into tablets, coated tablets, granules, powder, capsules or the like by a method that is well known in the art. Such additives can be those commonly employed in the art, including: excipients, e.g., saccharides (such as glucose, lactose, sucrose, brown sugar, sorbitol, mannitol, and starch), sodium chloride, calcium carbonate, kaolin, micro-crystalline cellulose, and silicic acid; binders, e.g., water, ethanol, propanol, sucrose solution, glucose solution, starch solution, gelatin solution, carboxymethylcellulose, hydroxypropylcellulose, hydroxypropylstarch, methylcellulose, ethylcellulose, shellac, calcium phosphate, and polyvinylpyrrolidone; disintegrators, e.g., dry starch, sodium alginate, powdered agar, sodium hydrogencarbonate, calcium carbonate, sodium lauryl sulfate, monoglycerol stearate, and lactose; lubricants, e.g., purified talc, stearate salts, borax, and polyethylene glycol; and corrigents, e.g., sucrose, bitter orange peel, citric acid, and tartaric acid.

When preparing a dressing for wound healing comprising the marine alga extract or the nanoparticles of chitosan-marine algae polysaccharides of low degree polymerization according to this invention, the dressing may optionally include ny of the conventional broad-spectrum or specific antibacterial agents and/or any of the conventional topical anesthetics. In addition, the dressing may further comprise other factors capable of promoting epithelial cell growth, such as fibrin, epidermal growth factor and/or human growth factor, various natural proteins extracted from the human body, various antibacterial/antimicrobial agents of Chinese and western medicine types, various anti-inflammatory agents, various autograft or xenograft skin materials, various animal skin materials or extracts, various metals and organic additives, or any combination thereof.

This invention will be further described by way of the following examples. However, it should be understood that the following examples are solely intended for the purpose of illustration only and should not be construed as limiting the invention in practice.

### EXAMPLES

### Experimental Materials

For the purpose of demonstration, marine algal materials belonging to Gracilaria lemaneformis, which were purchased from Tung-Kang, Taiwan, were used in all the experiments described below.

### General Procedures:

### A. Detection of the Total Sugar Content:

Detection of the total sugar content of an analyte sample was performed by the phenol-sulfuric acid colorimetric method (M. Dubois et al. (1956), Analytical Chemistry, 28:350-356). According to this method, sugars and derivatives thereof will react with concentrated sulfuric acid to form relatively stable orange-colored substances, which in turn may be detected by a spectrophotometer at a wavelength of 480 nm. The total sugar content of an analyte sample can thus be determined with reference to a standard calibration curve.

A 1 mL analyte sample was sequentially added with 0.025 mL 80% phenol and 2.5 mL concentrated sulfuric acid with even stirring, and the resultant mixture was allowed to stand for a few minutes. After cooling to room temperature, the absorbance of the mixture at a wavelength of 480 nm was detected by an ELISA Reader (Dynatech MR5000, Switzerland). The standard calibration curve was made using 0˜10 μg/mL galactose and mannose as standard sugars.

### B. Detection of the Reducing Sugar Content:

Detection of the reducing sugar content of an analyte sample was performed by the DNS method according to M. Oyaizu (1988) Nippon Shokuhin Kogyo Gakkaishi, 35:771-775 (G. L. Miller (1959), Analytical chemistry, 31:426-428). According to this method, dinitrosalicylic acid (DNS) will be reduced into an orange-red colored compound upon co-heating with a reducing sugar. Within a certain range of concentration, a linear relationship is shown between the amount of the reducing sugar and the absorbance of the orange-red colored compound. The total sugar content of an analyte sample can thus be determined by colorimetry.

A 0.1 mL analyte sample was sequentially added with 0.4 mL dd water and 0.4 mL of the DNS reagent (1% dinitrosalicylic acid+0.2% Phenol+0.05% Sodium sulfite+1% Sodium hydroxide in dd water (w/w)) with even stirring, and the resultant mixture was placed in a boiling water bath for 5 minutes. After addition of 0.4 mL dd water, the resultant mixture was allowed to cool to room temperature and then subjected to detection by a spectrophotometer (Hitachi U-2000) to determine the absorbance thereof at a wavelength of 540 nm. The reducing sugar content of an analyte sample can thus be determined with reference to a standard calibration curve, which is made using 0˜10 μg/mL galactose as standard sugar.

### C. Ultrafiltration:

Ultrafiltration was performed using an Amicon RA2000 ultrafiltration instrument equipped with Millipore Spiral-wound Membrane Cartridges S3Y1.

### D. Preparation of Human Melanoma Cells A375:

Human melanoma cells A375 were cultured with reference to W. K. Nahm et al. (2002), Journal of Dermatological Science, 28:152-158, and a 2002 master thesis, entitled “Immune modulating functions of yam polysaccharide from Dioscorea pseudojaponica,” by Ming-Chih Fang, Department of Food Science, National Taiwan Ocean University.

Firstly, the human melanoma cells A375 were cultivated in a culture plate containing DMEM supplemented with 5% FBS at 37° C. and 5% CO₂, and the growth thereof was observed under inverted microscope. Subculture was made when the cells were grown to form a confluent monolayer. The confluent monolayer cells were removed from the bottom of the culture plate by washing with phosphate-buffered saline (PBS) twice, followed by addition of 1 mL of trypsin (0.25% trypsin in Hank's balanced salt solution (HBSS)).

Prior to conducting the melanin synthesis inhibition assay, the cells were removed from the bottom of the culture plate by trypsin treatment, followed by centrifugation at 1,500 rpm for 5 minutes. The cells thus collected were adjusted with medium to a concentration suitable for use in the assay.

### E. Preparation of Human Skin Fibroblast Cells CCD-966SK:

The human skin fibroblast cells CCD-966SK (Lot-01175, obtained from the Food Industry Research and Development Institute (FIRDI)/the National Health Research Institute (NHRI) cell bank) were cultivated with Earle's Minimum Essential Medium (EMEM, HyClone) supplemented with 10% (v/v) FBS, 0.37% (w/v) NaHCO₃, two antibiotics (penicillin and streptomycin, each being used at a concentration of 100 units/mL), 0.1 mM of a non-essential amino acid solution (NEAA, HyClone), 1 mM sodium pyruvate and 0.03% L-glutamine and then incubated in a 37° C. incubator with 5% CO₂. Subculture was made when the cells were observed to become confluent under inverted microscope (approximately occurring at a time 2 to 3 days after cultivation). Prior to conducting the MTT assay, cells at the log phase were collected by trypsin treatment and centrifugation as described above, and were adjusted with medium to a concentration suitable for use in the assay.

### F. Particle Size Analysis:

The particle size of nanoparticles were primarily detected by a laser scattering instrument, with the aid of scanning electron microscopy (SEM) in the observation of particle size and structure.

### 1. Detection by a Laser Scattering Instrument:

A 3 mL analyte sample was placed into a sample tube. The laser scattering instrument (Malvern 4700, Malvern instrument, U.K.) was set to have an incident wavelength of 633 nm and then used to detect the scattering light intensity of the sample at an angle of 90° and at a temperature of 30±0.1° C. The scattering light intensity can be converted into a diffusion coefficient, which in turn can be introduced into the Stoke-Einstein equation to calculate the particle size (T. Banerjee et al. (2002), International Journal of Pharmaceutics, 243:93-105; M. L. Tsaih and R. H. Chen (1997), Journal of Applied Polymer Science, 71:1905-1913; a 2003 master thesis, entitled “Preparation of Chitosan Nanoparticles and Their Application on the Controlled Release of Erythromycin,” by Guo-Xuan Fan, Department of Food Science, National Taiwan Ocean University).

### B. Detection by Scanning Electron Microscopy (SEM).

A carbon gel was placed on a stage and a 20 μL analyte sample was deposited thereon, followed by drying in an oven for 1 day. After being coated with a layer of gold on the surface thereof by an ion coater, the sample was examined by a scanning electron microscope (Hitachi S-4100 & Hitachi S-4700)(15 kV, 30K˜100K magnification)(F. L. Mi et al., (2002), Biomaterials, 23:181-191).

### Example 1

### The Influence of Reaction Time of Acid Hydrolysis Upon the Degree of Polymerization of Marine Algal Polysaccharides of Gracilaria lemaneformis

This example was conducted to determine the influence of reaction time of acid hydrolysis upon the degree of polymerization of marine algal polysaccharides contained in an extract solution of Gracilaria lemaneformis.

Preparation of Hot-Water Extract of Gracilaria lemaneformis:

Marine algal material of Gracilaria lemaneformis that had been washed, dried and cut into small pieces was immersed in 20- to 30-fold dd water (alternatively, the dried algal bodies of Gracilaria lemaneformis in an appropriate amount were cut into small pieces, washed with dd water, and immersed in dd water for 1˜3 hours to soften the algal bodies and to remove undesired impurities, followed by addition of 50-fold dd water). The resultant aqueous solution containing the marine algal material of Gracilaria lemaneformis was subsequently subjected to heat extraction using an oil bath inside a 100° C. temperature control tank for 6 hours with continuous stirring. Thereafter, the debris of the heat-extracted algal bodies was removed by gauze filtration, giving an aqueous hot-water extract of Gracilaria lemaneformis that had marine algal polysaccharides dissolved therein. Alternatively, the aqueous hot-water extract thus obtained could be lyophilized for later use.

Acetic Acid Hydrolysis of Hot-Water Extract of Gracilaria lemaneformis:

The aqueous hot-water extract as obtained above was admixed with 10% acetic acid or, alternatively, the lyophilized product of the aqueous hot-water extract as obtained above was dissolved in a preliminarily prepared acetic acid solution. The resultant aqueous solution that had an appropriate concentration of acetic acid was then allowed to stand overnight to permit the swelling of the marine algal polysaccharides contained therein.

Any of the resultant acetic acid solutions containing swelled marine algal polysaccharides was subjected to heat extraction using a water bath inside a temperature control tank set at 90° C. with continuous stirring. An aliquot of the solution was sampled every hour for 10 hours. The total sugar content and the reducing sugar content of each of the samples thus taken were respectively detected according to the methods set forth in Item A and Item B of the General procedures described above. The average degree of polymerization of the marine algal polysaccharides contained in the sample could be calculated by dividing the value of the detected total sugar content with the value of the detected reducing total sugar content, i.e., the ratio of total sugar content to the reducing sugar content.

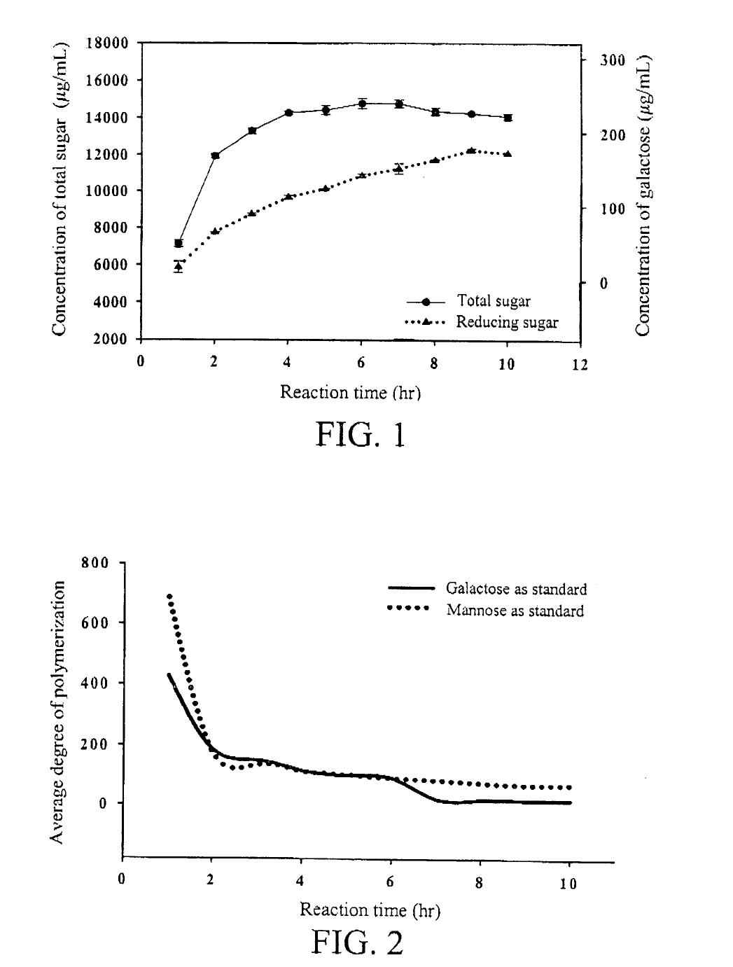

The obtained results are shown in FIGS. 1 and 2, in which all the results are represented by mean ±S.D. (n=—4).

### Detection of Specific Viscosity:

In conducting the assay, a capillary viscometer with a smaller inner diameter (Cannon-Fenske, No 100) was immersed in a constant-temperature bath inside a temperature control tank (Tamson, TMV 40, Sweden) equipped with a temperature controller (Firstek, B403, Taipei) and maintained at 30±0.05° C.

Prior to the specific viscosity detection, each of the samples taken at different time intervals of the acetic acid hydrolysis was neutralized by NaOH, while the capillary viscometer was rinsed with dd water for several times. Subsequently, the capillary viscometer was rinsed with the neutralized sample once and then placed into the 30° C. water bath for detection.

The time needed for each of the neutralized samples to pass through the capillary viscometer was recorded. The specific viscosity (ηₛₚ)=the time needed for a solution to pass through the capillary viscometer/the time needed for a solvent to pass through the capillary viscometer.

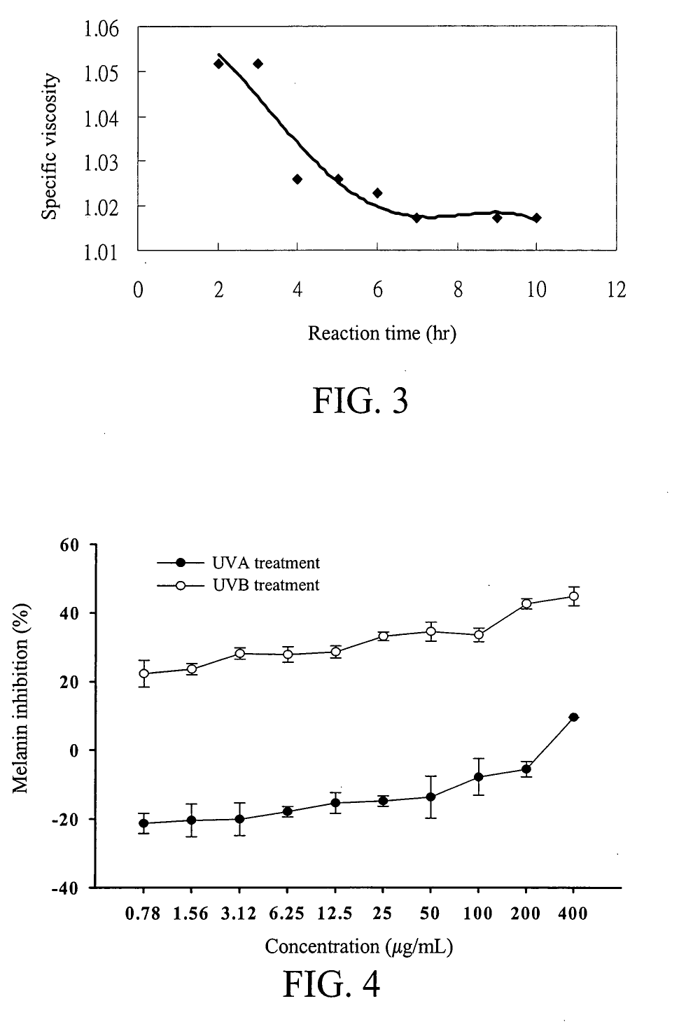

The obtained results are shown in FIG. 3, in which all the results are represented by mean ±S.D. (n=3).

### Results:

It can be seen from FIG. 1 that the reducing sugar content of a sample tends to increase in a linear relationship with the reaction time of acid hydrolysis. The total sugar content increased in a linear relationship within the first 5 hours and then slightly lowered down.

Referring to FIG. 2, the acid hydrolysis resulted in a significant decrease of the degree of polymerization of the marine algal polysaccharides contained in the hot-water extract of Gracilaria lemaneformis within the first two hours, and then the decrease of the degree of polymerization of marine algal polysaccharides was lowered down slowly within the 2nd to 7th hours. After 7 hours, the average degree of polymerization of marine algal polysaccharides gradually reached a constant value of around 20 saccharide units. Therefore, a better acid hydrolysis of the marine algal polysaccharides can be achieved at a reaction time in the range of 5 to 7 hours.

The aqueous hot-water extract of Gracilaria lemaneformis has a high viscosity. Therefore, the degrees of hydrolysis of the marine algal polysaccharides contained in the samples may be predicted based on the detected viscosities of the samples. The lower the specific viscosity is, the higher the degrees of polysaccharide hydrolysis will be. It can be seen from FIG. 3 that the specific viscosity of samples has a significant drop within the 2nd to 6th hours of acid hydrolysis, and no significant change occurs during the 6th to 10th hours of acid hydrolysis. This further evidences the usage of acid hydrolysis.

### Example 2

### Preparation of Marine Algal Extract of Gracilaria lemaneformis Comprising Marine Algal Polysaccharides of Low Degree Polymerization

The aqueous hot-water extract as obtained in Example 1 above was admixed with 10% acetic acid or, alternatively, the lyophilized product of the aqueous hot-water extract as obtained in Example 1 above was dissolved in a preliminarily prepared acetic acid solution. The resultant aqueous solution that had an appropriate concentration of acetic acid was then allowed to stand overnight to permit the swelling of the marine algal polysaccharides contained therein.

Any of the resultant acetic acid solutions containing swelled marine algal polysaccharides was subjected to heat extraction using a water bath inside a temperature control tank set at 90° C. with continuous stirring.

After the swelled marine algal polysaccharides were heat-hydrolyzed to appropriate molecular weight, the resultant solution was subjected to a ultrafiltration treatment as described in Item C of the section of “General procedures,” so as to remove marine algal polysaccharides of very low molecular weight or those of high molecular weight, as well as salt compounds of low molecular weight. Alternatively, the resultant solution after ultrafiltration was lyophilized to give a solid extract product comprising marine algal polysaccharides of low degree polymerization.

### Example 3

### Preparation of Marine Algal Extract of Gracilaria lemaneformis Comprising Marine Algal Polysaccharides of Low Degree Polymerization

The aqueous hot-water extract as obtained in Example 1 above was admixed with 10% acetic acid or, alternatively, the lyophilized product of the aqueous hot-water extract as obtained in Example 1 above was dissolved in a preliminarily prepared acetic acid solution. The resultant aqueous solution that had an appropriate concentration of acetic acid was then allowed to stand overnight to permit the swelling of the marine algal polysaccharides contained therein.

Any of the resultant acetic acid solutions containing swelled marine algal polysaccharides was subjected to a ultrasonication treatment at 90° C. for 1 to 8 hours, so as to facilitate the hydrolysis of marine algal polysaccharides to an appropriate molecular weight. Thereafter, the resultant solution was subjected to a ultrafiltration treatment as described in Item C of the section of “General procedures,” so as to remove marine algal polysaccharides of very low molecular weight or those of high molecular weight, as well as salt compounds of low molecular weight. Alternatively, the resultant solution after ultrafiltration was lyophilized to give a solid extract product comprising marine algal polysaccharides of low degree polymerization.

### Example 4

### The Effect of Marine Algal Extract of Gracilaria lemaneformis Comprising Marine Algal Polysaccharides of Low Degree Polymerization in Inhibiting Melanin Production by Human Melanoma Cells A375

In order to explore the potential of the marine algal extract comprising marine algal polysaccharides of low degree polymerization according to this invention in inhibiting melanin production, a lyophilized powder product of the marine algal extract of Gracilaria lemaneformis prepared according to Example 2 and comprising marine algal polysaccharides having a molecular weight in the range of 1×10³ Daltons to 5×10³ Daltons was employed in this example, and the effect of said product in inhibiting melanin synthesis by human melanoma cells A375 was detected according to the methodology reported in a 2002 master thesis, entitled “The study of the efficacy of melanin inhibitors and moisturizers for skin,” by Yi-Shyan Chen, Department of Applied Chemistry, Providence University.

### Experimental Procedures:

To a 96-well culture plate, each well was inoculated with 1×10⁵ melanoma cells, followed by addition of 2-fold serial dilutions of marine algal extract comprising marine algal polysaccharides of low degree polymerization according to this invention (0.78˜400 μg/mL) as prepared in the basic culture medium. The basic culture medium alone was used as a control group. The culture plate was then incubated in a 37° C. incubator with 5% CO₂ for 3 days. After Ultraviolet (UV) irradiation with either Ultraviolet A (UVA, 365 nm) or Ultraviolet B (UVB, 302 nm) at a light intensity of 1.1 mw/cm² for 15 minutes, the culture plate was cultivated for a further 24 hours, followed by addition of 100 μL of a 1 N NaOH solution to each well with gentle agitation. Thereafter, the culture plate was subjected to detection by a spectrophotometer at a wavelength of 400 nm, and the detected absorbance was introduced into the following equation to calculate melanin inhibition rate.

Melanin inhibition(%)=[(A₄₀₀ of control−A₄₀₀ of sample)/A₄₀₀ of control]×100

### Results:

As can be seen from FIG. 4, the marine algal extract comprising marine algal polysaccharides of low degree polymerization according to this invention is capable of inhibiting melanin synthesis by human melanoma cells A375, the inhibitory effect of the extract having a linear relationship with the concentration of the extract used. In addition, the extract is more effective in inhibiting melanin synthesis caused by UVB irradiation, in which a melanin inhibition rate of 45% was reached at a concentration of 400 μg/mL and the melanin inhibition rate tends to increase with an increase of the concentration of the extract.

### Example 5

### The Effect of Marine Algal Extract of Gracilaria lemaneformis Comprising Marine Algal Polysaccharides of Low Degree Polymerization in Scavenging DPPH Radicals

This example evaluates the ability of the marine algal extract comprising marine algal polysaccharides of low degree polymerization according to this invention in scavenging α,α-diphenyl-β-picryhydrazyl (DPPH) radicals, in which the marine algal extract tested herein was prepared in the same way as described in Example 4 above.

### Experimental Procedures:

The DPPH radical scavenging activity was detected based on the methodologies reported in F. Bonina et al. (1998), International Journal of Cosmetic Science, 20:331-342 and K. Shimada et al. (1992), Journal of Agricultural and Food Chemistry, 40:945-948.

Briefly, 4 mL of a solution containing the marine algal extract comprising marine algal polysaccharides of low degree polymerization according to this invention in various concentrations (0.2%, 0.4%, 0.6%, 0.8% and 1.0% by weight) was admixed with 1 mL of a freshly prepared ethanol or water solution containing 0.2 mM DPPH. After mixing well, the resultant mixture was allowed to stand for 30 minutes and then detected by a spectrophotometer (Hitachi U-2000) to determine the absorbance thereof at a wavelength of 517 nm. 4 mL of an ethanol or water solution was used as the blank control group, and a 1.0% BHT solution was used as the positive control group.

The DPPH radical scavenging activity was determined by introducing the detected absorbance of a sample into the following equation, in which the lower the detected absorbance was, the stronger the DPPH radical scavenging activity of the sample would be.

Scavenging rate=[1−(A₅₁₇ of the sample/A₅₁₇ of the blank control)]×100%

### Results:

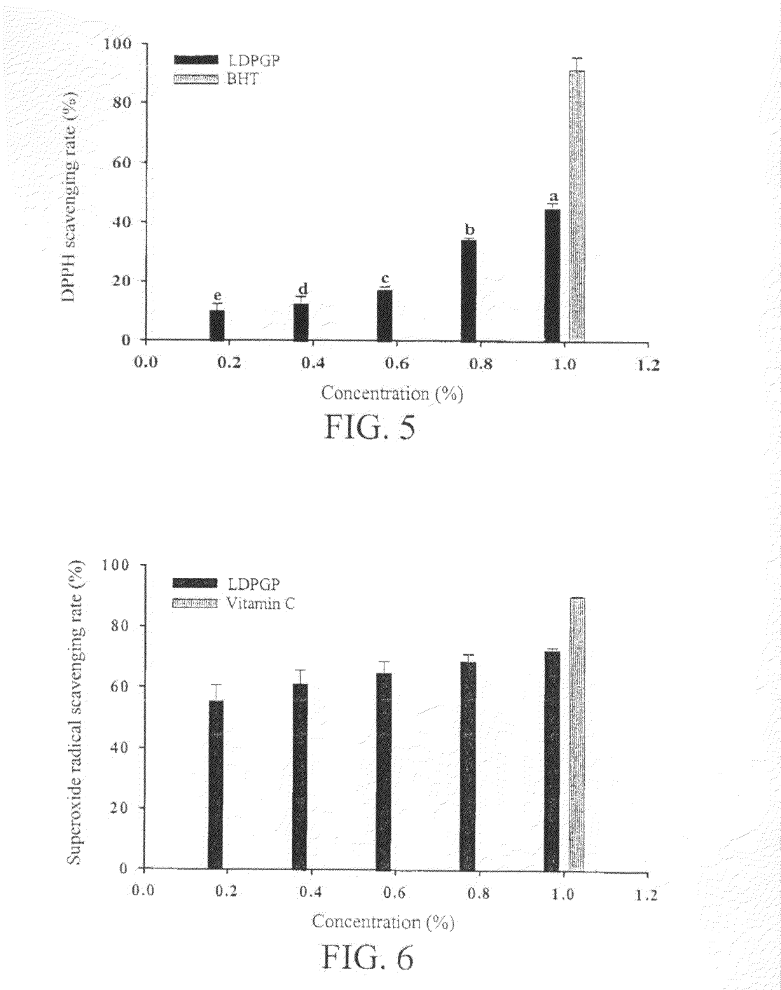

As can be seen from FIG. 5, the DPPH radical scavenging activity of the marine algal extract comprising marine algal polysaccharides of low degree polymerization according to this invention has a linear relationship with the concentration of the extract falling within the range of the used concentrations thereof (i.e., within 1% by weight), and tends to increase with an increase of the concentration of the extract.

### Example 6

### The Effect of Marine Algal Extract of Gracilaria lemaneformis Comprising Marine Algal Polysaccharides of Low Degree Polymerization in Scavenging Superoxide Radicals

This example evaluates the ability of the marine algal extract comprising marine algal polysaccharides of low degree polymerization according to this invention in scavenging superoxide radicals, in which the marine algal extract tested herein was prepared in the same way as described in Example 4 above. The assay was conducted based on the methodologies reported in J. Robak and R. J. Gryglewski (1988), Biochemical Pharmacology, 17:837-841 and F. Liu and TB. Ng (1999), Life Sciences, 66:725-735.

### Experimental Procedures:

A 0.1 M phosphate buffer (pH 7.4) was used to prepare a 120 μM PMS solution, a 936 μM NADH solution and a 300 μM NBT solution. Meanwhile, the marine algal extract comprising marine algal polysaccharides of low degree polymerization according to this invention was prepared in a 0.1 M phosphate buffer (pH 7.4) in various concentrations (0.2%, 0.4%, 0.6%, 0.8% and 1.0% by weight). Aliquots (1 mL) of the phosphate-buffered solution containing the marine algal extract thus prepared were sequentially added with the PMS, NADH and NBT solutions each in a volume of 1 mL. After mixing well, the resultant mixture was allowed to stand for 5 minutes at room temperature and then detected by a spectrophotometer (Hitachi U-2000) to determine the absorbance thereof at a wavelength of 560 nm. 1 mL of the phosphate buffer solution was used as the blank control group, and 1 mL of a 1.0% vitamin C solution was used as the positive control group.

The superoxide radical scavenging activity was determined by introducing the detected absorbance of a sample into the following equation, in which the lower the detected absorbance was, the stronger the superoxide radical scavenging activity of the sample would be.

Scavenging rate=[1−(A₅₆₀ of the sample/A₅₆₀ of the blank control)]×100%

### Results:

As can be seen from FIG. 6, the superoxide radical scavenging activity of the marine algal extract comprising marine algal polysaccharides of low degree polymerization according to this invention has a linear relationship with the concentration of the extract falling within the range of the used concentrations thereof (i.e., within 1% by weight), and tends to increase with an increase of the concentration of the extract.

### Example 7

### Detection of the Reducing Power of Marine Algal Extract of Gracilaria lemaneformis Comprising Marine Algal Polysaccharides of Low Degree Polymerization

This example evaluates the reducing power of the marine algal extract comprising marine algal polysaccharides of low degree polymerization according to this invention, in which the marine algal extract tested herein was prepared in the same way as described in Example 4 above. The assay was conducted based on the methodologies reported in M. Oyaizu (1988), Nippon Shokuhin Kogyo Gakkaishi, 35:771-775.

### Experimental Procedures:

Aliquots (2 mL) of samples were admixed with 2 mL of 0.2 M phosphate buffered solution (pH 6.5) and 2 mL of 1% potassium ferrocyanide and placed in a 50° C. water bath for 20 minutes. Thereafter, the resultant mixture was quickly cooled and added with 2 mL of a 10% trichloroacetic acid solution. After mixing well, 2 mL of the resultant mixture was taken and added with 2 mL distilled water and 0.4 mL of a 0.1% ferric chloride solution. After mixing well for 10 minutes, the resultant mixture was detected by a spectrophotometer (Hitachi U-2000) to determine the absorbance thereof at a wavelength of 700 nm. The higher the detected absorbance was, the higher the reducing power of the sample would be. A 1.0% vitamin C solution was used as a control group.

### Results:

The reducing power assay is based on the production of Prussian blue, which is produced due to the reduction of Fe(CN)₆³⁺ to Fe(CN)₅²⁺, and which exhibits an absorbance at a wavelength of 700 nm. As can be seen from FIG. 7, the detected absorbance increases with an increase of the concentration of the marine algal extract comprising marine algal polysaccharides of low degree polymerization according to this invention. The obtained results indicate that the reducing power of the extract tends to increase with an increase of the concentration of the extract.

### Example 8

### The Effect of Marine Algal Extract of Gracilaria lemaneformis Comprising Marine Algal Polysaccharides of Low Degree Polymerization Upon the Cell Proliferation of Human Skin Fibroblast Cell Line CCD-966SK

This example evaluates the effect of the marine algal extract comprising marine algal polysaccharides of low degree polymerization according to this invention upon the cell proliferation of human skin fibroblast cells CCD-966SK (Lot-01175, obtained from the Food Industry Research and Development Institute (FIRDI)/the National Health Research Institute (NHRI) cell bank), in which various concentrations (0.3125˜200 μg/mL) of the extract prepared in the same way as described in Example 4 above were tested, and the cell proliferation rate was determined by the MTT method after cultivation of the cells for 48 hour (B. J. Phillips (1996), Toxicology in vitro, 10:69-76; H. Jiao et al. (1992), Journal of Immunological Methods, 153:265-266).

### Experimental Procedures:

The marine algal extract comprising marine algal polysaccharides of low degree polymerization according to this invention was dissolved in the culture medium containing 1% FBS to a concentration of 1 mg/mL. The resultant mixture was filtrated through a 0.22 μm filter using a plastic sterile syringe. Various amounts of the filtrated mixture were added into each well of a 96-well culture plate, followed by addition of the culture medium, so that each well contained a test fluid of 25 μL/mL with 10 different concentrations of the extract ranging from 1 to 0.001 mg/mL. Thereafter, cells at the log phase were adjusted to a concentration of 1×10⁵ cells/mL with medium, and 100 μL of the cells was added into each well. The culture plate was incubated in a 37° C. incubator with 5% CO₂. After cultivation for 2 days, the cells were detected by the MTT method to determine the cell viability rate thereof.

### Results:

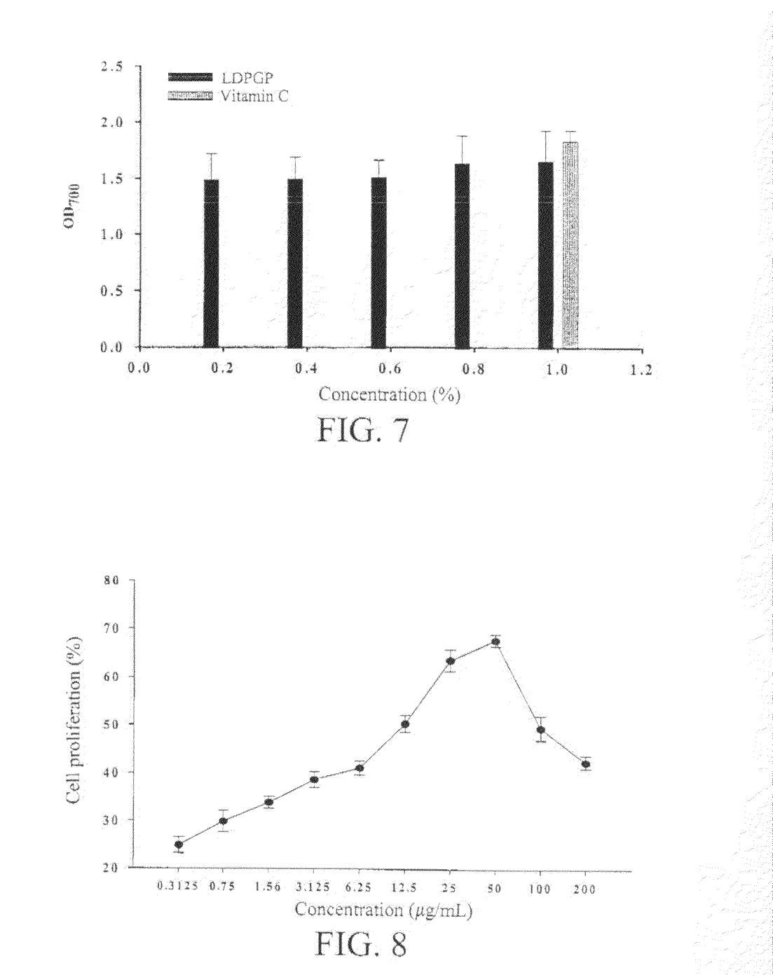

As can be seen from FIG. 8, the marine algal extract comprising marine algal polysaccharides of low degree polymerization according to this invention is capable of promoting the cell proliferation of human skin fibroblast cells CCD-966SK within the used concentration of 200 μg/mL. Noting that fibroblast cells in human skins have the functions of secreting collagen and maintaining skin elasticity, it is contemplated that the marine algal extract comprising marine algal polysaccharides of low degree polymerization according to this invention, which is capable of promoting the cell proliferation of fibroblast cells, should also be useful in improving skin elasticity and thus reach the effect of anti-aging of skin.

### Example 9

### The Effect of Marine Algal Extract of Gracilaria lemaneformis Comprising Marine Algal Polysaccharides of Low Degree Polymerization Upon Collagen Synthesis

This example analyzes the effect of the marine algal extract comprising marine algal polysaccharides of low degree polymerization according to this invention upon collagen synthesis by human skin fibroblast cells CCD-966SK, in which various concentrations (1.5625, 3.75, 7.8, 15.625, 31.25, 62.5, 125 and 250 μg/mL) of the extract were tested.

### Experimental Procedures:

Collagen synthesis analysis was performed according to the following references: T. Yamamoto and K. Nishioka (2001), Journal of Investigative Dermatology, 117:999-1001, Y. Y. Li et al. (2001), Circulation, 104:1147-1152, and K. Blease et al. (2002), American Journal of Pathology, 160:481-490.

To a 96-well culture plate, each well was inoculated with 2×10⁴ fibroblast cells, followed by addition of medium containing the marine algal extract comprising marine algal polysaccharides of low degree polymerization according to this invention in various concentrations. The culture plate was incubated in a 37° C. incubator with 5% CO₂ for 48 hours. The basal medium was used as a control group.

To determine the collagen content thereof, the cell culture as cultivated in each well was collected and tested using a Sircol Collagen Assay Kit (S1000, Biocolor Ltd., Belfast, Ireland). Aliquots (50 μL) of the samples were placed into a 1.5 mL microcentrifuge tube in combination with reagent blanks (0.5 M acetic acid) to a volume of 100 μL. As a control group, a basal medium of the same volume was used in place of the sample. To each tube, 1 mL Reagent A (Sircol dye reagent) was added and mixed for 30 minutes, followed by centrifugation at 5,000 g for 5 minutes. After removal of the supernatant, 1 mL Reagent B (Alkali reagent) was added and mixed well by agitation. The resultant mixture contained in each tube was subsequently detected by a spectrophotometer (Hitachi U-2000) to determine the absorbance thereof at a wavelength of 540 nm. The standard calibration curve was made using Collagen Standards (S1010) containing collagen in amounts of 1, 2, 5, 6.25 and 12.5 μg, respectively. After regression analysis of the results of the Collagen Standards, a linear equation was obtained and the collagen content of a test sample as expressed by μg/mL could be determined according to said equation.

### Results:

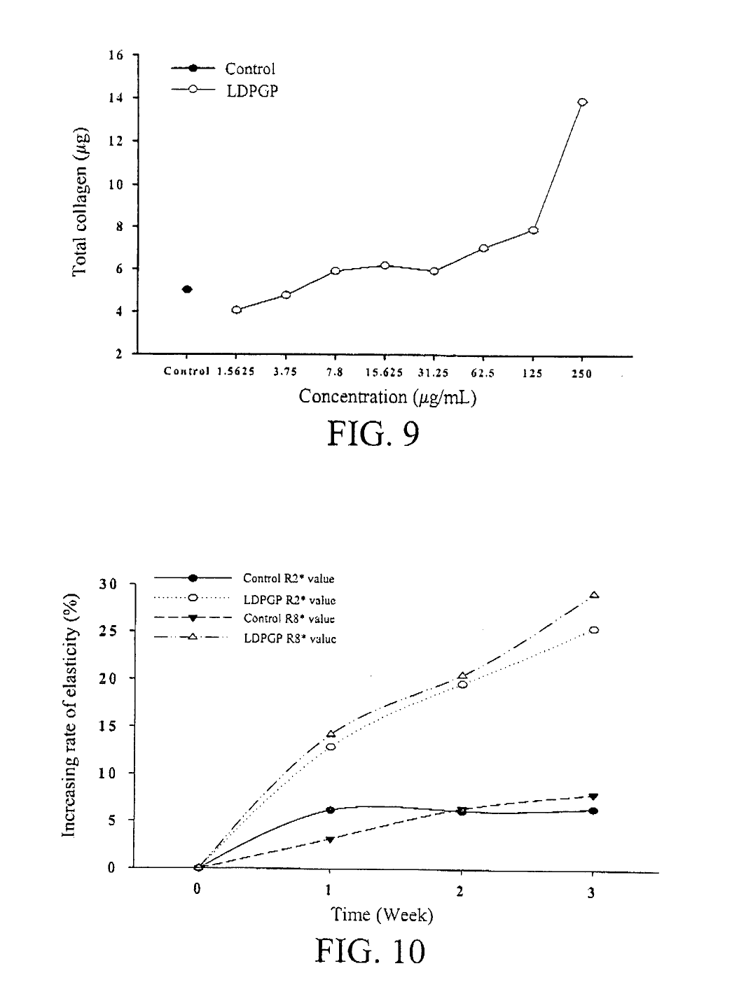

As can be seen from FIG. 9, higher amounts of collagen were produced in treatment groups in which the human skin fibroblast cells CCD-966SK were cultivated in cell culture medium containing the marine algal extract comprising marine algal polysaccharides of low degree polymerization according to this invention at a concentration higher than 7.8 μg/mL, as compared to that of the control group. In addition, the collagen content tends to increase with an increase of the concentration of the extract, indicating that the marine algal extract comprising marine algal polysaccharides of low degree polymerization according to this invention is effective in promoting collagen synthesis.

### Example 10

### Usage of Marine Algal Extract of Gracilaria lemaneformis Comprising Marine Algal Polysaccharides of Low Degree Polymerization in Skin Care Cosmetic Product

### Experimental Procedures:

Firstly, an aqueous phase (A) and an oil phase (B) were prepared according to the basic formulation shown in the following Table 4, in which the marine algal extract comprising marine algal polysaccharides of low degree polymerization according to this invention was used as an ingredient of the phase A. The phase A and the phase B were separately placed into a 70° C. water bath. After being completely dissolved by heating, the two phases were allowed to stand at that temperature for a further 20 minutes, and then removed from the water bath. The phase B was slowly added into the phase A to cause emulsification, followed by homogenization with a homogenizer (PT-MR 3000, Polytron). After cooling, a vital cream containing the marine algal extract comprising marine algal polysaccharides of low degree polymerization according to this invention was obtained. A cream of the same formulation but not containing the extract was used as a control group.

TABLE 4
The basic formulation of a vital cream containing the marine algal
extract comprising marine algal polysaccharides of low degree
polymerization according to this invention

| Phases | Ingredients | Used amounts (g) |
|---|---|---|
| A (aqueous phase) | Water | 76.35 |
| | KOH | 0.2 |
| | Propylene glycol | 5.0 |
| | Methyl paraben (M.P) | 0.1 |
| | The marine algal extract | 0.25 |
| B (oil phase) | Stearic acid | 5.0 |
| | Cetyl alcohol | 4.0 |
| | Wickenol 158 | 6.0 |
| | P.P. | 0.1 |
| | GMS 1330 surfactant | 1 |

The variation of skin elasticity of the lower arm of each volunteer was evaluated by the suction method. The inner side of a lower arm of a volunteer was marked with two areas having a size of 25 cm², one area being the experimental group and the other one being the control group. The vital cream and the control cream each in an amount of 0.2 grams were applied to the marked areas located at the inner side of each volunteer's upper/lower arm once a day for 3 weeks. At 20° C. and a relative humidity (RA) of 60˜65%, the skin elasticity values of each volunteer's lower arm skin were measured once every week as of week 0. The higher the measured R2 value is, the better the skin elasticity will be. Besides, the skin may have a higher elasticity if the measured R8 value is approaching a value of 1. The increase rate of skin elasticity corresponds to the increase rates of the R2 value and the R8 value and can be calculated by the following equation:

The increase rate of skin elasticity=[(the measured value at week X−the measured value at week 0)/The measured value at week 0]×100%

in which the R2 and R8 values were directly obtained from the skin elasticity values of the test skins as measured by a Cutometer SEM 575.

The higher the calculated increase rate is, the better the skin elasticity will be.

### Results:

FIG. 10 shows the variation of skin elasticity caused by the application of a vital cream containing the marine algal extract comprising marine algal polysaccharides of low degree polymerization according to this invention or a control cream to the marked areas located on the inner side of each volunteer's lower arm for three weeks. The results reveal that the R2 value significantly increases after a continuous application of the vital cream for 1 week, and continues to increase after 2 and 3 weeks of application. In addition, a significant increase of the R8 value appears after a continuous application of the vital cream for 2 weeks. The obtained results reveal that the marine algal extract comprising marine algal polysaccharides of low degree polymerization according to this invention is effective in improving skin elasticity and, hence, is a promising agent for anti-aging of skin.

### Example 11

### Preparation of Nanoparticles of Chitosan-Marine Algal Polysaccharides of Low Degree Polymerization

This example illustrates the preparation of nanoparticles of chitosan-marine algal polysaccharides of low degree polymerization via ionic gelation. Briefly, a chitosan solution in a suitable concentration was reacted with a solution containing a marine algal extract comprising marine algal polysaccharides according to this invention. The resultant mixture was subjected to a ultrasonication treatment to facilitate electrostatic interaction between positively and negatively charged substances, leading to the formation of nanoparticles of chitosan-marine algal polysaccharides of low degree polymerization.

A 20 mL solution of chitosan prepared in 0.05% acetic acid solution and having a suitable concentration was reacted with 60 mL of an aqueous solution containing the marine algal extract of Gracilaria lemaneformis (LDPGP) prepared according to Example 2 in a concentration of 0.1% (w/w). The resultant mixture was sonicated at 60 W to cause the production of nanoparticles of chitosan-marine algal polysaccharides of low degree polymerization, followed by centrifugation at 10,000 rpm (CR21, Hitachi, Ltd., Japan) for 30 minutes. The resultant supernatant containing the nanoparticles was collected and stored at low temperature (4±1° C.) or room temperature (30±1° C.) or high temperature (50±1° C.) for later use or, as an alternative, the supernatant could be lyophilized to give lyophilized nanoparticles for later use.

### Example 12

### The Influence of the Concentration of Chitosan Upon the Average Particle Size of Nanoparticles of Chitosan-Marine Algal Polysaccharides of Low Degree Polymerization

In order to understand the influence of the concentration of chitosan upon the average particle size of nanoparticles of chitosan-marine algal polysaccharides of low degree polymerization, this example used a laser scattering instrument to determine the particle sizes of nanoparticles of chitosan-marine algal polysaccharides of low degree polymerization prepared according to the process set forth in Example 11 and using different concentrations of chitosan.

### Experimental Procedures:

Nanoparticles of chitosan-marine algal polysaccharides of low degree polymerization were prepared according to the procedures set forth in Example 11, in which the marine algal extract of Gracilaria lemaneformis was used at a concentration of 0.1%, and chitosan was used at a concentration of 0.001, 0.01, 0.1 and 1% (w/w). The nanoparticles of chitosan-marine algal polysaccharides of low degree polymerization thus obtained were subjected to particle size analysis.

### Results:

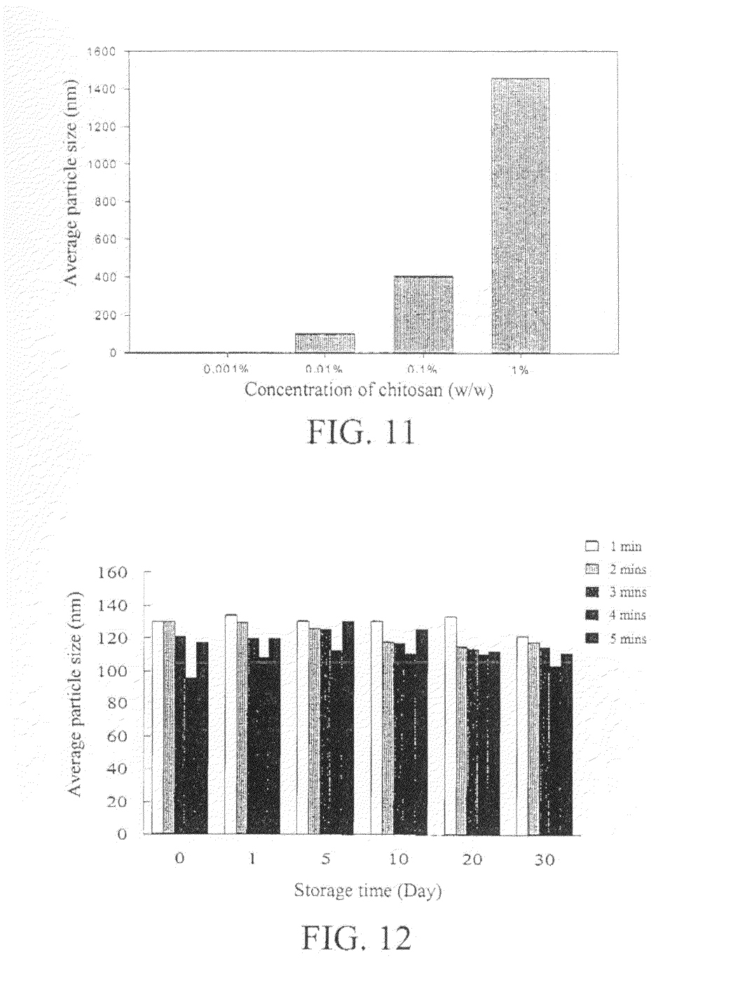

FIG. 11 shows that the average particle size of nanoparticles of chitosan-marine algal polysaccharides of low degree polymerization varied with the concentrations of chitosan. When chitosan was used at a concentration of 1% by weight, the nanoparticles thus obtained had an average particle size of 1456 nm; when chitosan was used at a concentration of 0.1% by weight, the nanoparticles thus obtained had an average particle size of 403 nm; when chitosan was used at a concentration of 0.01% by weight, the nanoparticles thus obtained had an average particle size of 105 nm; and when the concentration of chitosan was reduced to 0.001% by weight, the average particle size of the nanoparticles thus obtained was not detectable. Evidently, the concentration of chitosan influences the average particle size of the nanoparticles prepared therefrom. Within a suitable range of concentration, the lower the concentration of chitosan is, the smaller the average particle size of the nanoparticles of chitosan-marine algal polysaccharides of low degree polymerization will be.

### Example 13

### The Influences of the Ultrasonication Time, the Storage Time and the Storage Temperature Upon the Average Particle Size of Nanoparticles of Chitosan-Marine Algal Polysaccharides of Low Degree Polymerization

In order to understand the influences of the ultrasonication time, the storage time and the storage temperature upon the average particle size of nanoparticles of chitosan-marine algal polysaccharides of low degree polymerization, in this example, nanoparticles of chitosan-marine algal polysaccharides of low degree polymerization, which were prepared at different times of ultrasonication and stored at different temperatures for different storage times, were subjected to particle size analysis.

### A. The Influence of the Ultrasonication Time Upon the Average Particle Size of Nanoparticles of Chitosan-Marine Algal Polysaccharides of Low Degree Polymerization

### Experimental Procedures:

Nanoparticles of chitosan-marine algal polysaccharides of low degree polymerization were prepared according to the procedures set forth in Example 11, in which the marine algal extract of Gracilaria lemaneformis and chitosan were used at a concentration of 0.01% by weight, respectively, and the reaction mixture was subjected to different times of ultrasonication (1, 2, 3, 4 and 5 minutes). Supernatants as collected from different experimental groups were stored at different temperatures of 4° C., 30° C. and 50° C. for different times of 0, 1, 5, 10, 20 and 30 days and then subjected to particle size analysis as described above.

### Results:

FIG. 12 shows the variation of average particle size of nanoparticles of chitosan-marine algal polysaccharides of low degree polymerization as prepared above with a ultrasonication time of 4 minutes and stored at room temperature for different times of 0, 1, 5, 10, 20 and 30 days. The results reveal that nanoparticles of chitosan-marine algal polysaccharides of low degree polymerization which were prepared at an ultrasonication time of 4 minutes and stored for 30 days had an average particle size of smallest value.

### B. The Influences of the Storage Temperature and Time Upon the Average Particle Size of Nanoparticles of Chitosan-Marine Algal Polysaccharides of Low Degree Polymerization

### Experimental Procedures:

Nanoparticles of chitosan-marine algal polysaccharides of low degree polymerization which were prepared at an ultrasonication time of 4 minutes in the above-described section A were stored at low temperature (4±1° C.), room temperature (30±1° C.) and high temperature (50±1° C.) for different times of 0, 1, 5, 10, 20 and 30 days and then subjected to particle size analysis as described above.

### Results:

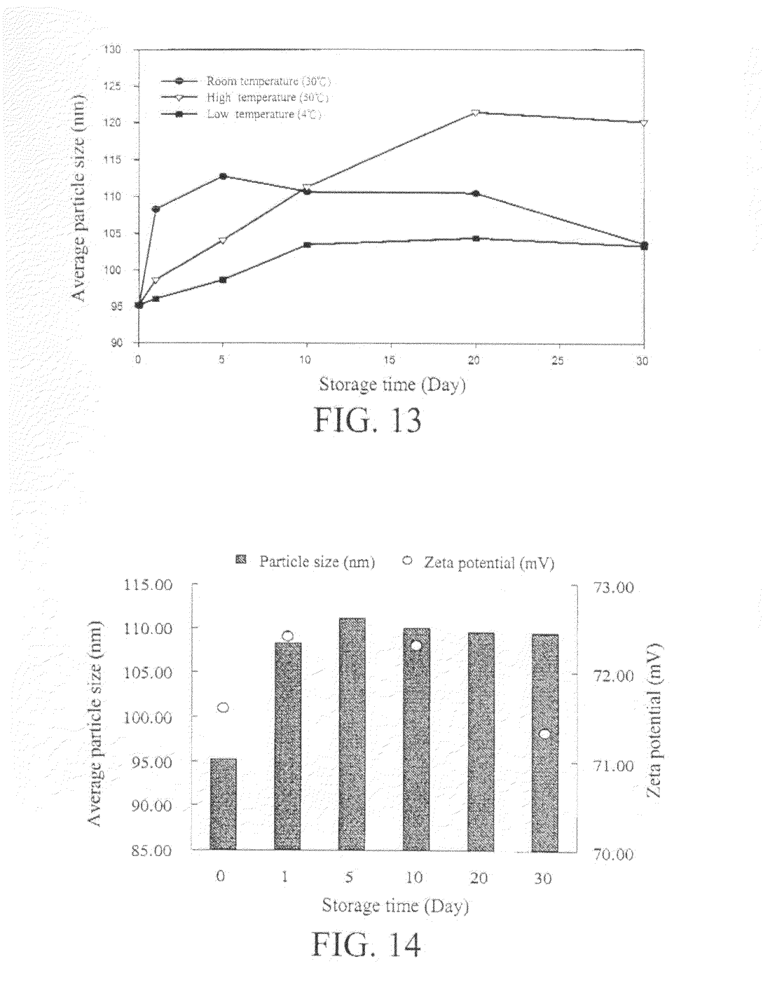

FIG. 13 shows the variation of average particle size of nanoparticles of chitosan-marine algal polysaccharides of low degree polymerization as prepared above with a ultrasonication time of 4 minutes and stored at three different temperatures for different times of 0, 1, 5, 10, 20 and 30 days. The results reveal that the particle sizes of the nanoparticles are slightly increased with time at the three different storage temperatures and, after 10 days of storage, no increase of the particle size was observed for nanoparticles stored at low temperature and room temperature, but the particle sizes of the nanoparticles stored at high temperature tends to keep increasing. According to the value of average particle size detected on Day 30, the particle size of nanoparticles stored at high temperature stopped increasing after 20 days of storage. A possible explanation for the observed larger particle size of nanoparticles stored at high temperature may be that high temperature increases the interaction among particles, causing the hydration and agglutination of nanoparticles. On the other hand, a possible explanation for the observed smaller particle size of nanoparticles stored at low temperature and room temperature may be that: firstly, dehydration results in a reduction of the particle size of nanoparticles; and secondly, the nanoparticles agglutinate to form large particles that will precipitate at the bottom of solution, so that only small particles are detected to give the observed smaller particle size.

### Example 14

### The Storage Stability of Nanoparticles of Chitosan-Marine Algal Polysaccharides of Low Degree Polymerization

Generally, when a substance comes into contact with water or any other organic solvent, the surface thereof will become electrically charged due to the adsorption of ions thereto (see a 2006 master thesis, entitled “Effects of Ultrasonic Radiation Treatment and Mechanical Shear Force on the Particle Size and Storage Stability of Chitosan Nanoparticles,” by Shi-Wei Bai, Department of Food Science, National Taiwan Ocean University). The surface charge property of colloids is the same as that of common electrolytes. The electric double layer is a fixed layer (or called “Stern layer”) adsorbed on the surface of a colloidal particle. When the colloidal particle exerts relative movement to the environment where it is present, the fixed layer will move together therewith, and the potential existing between the surface of the fixed layer and its outer diffusion layer is called “zeta potential.” The higher the zeta potential is, the greater the repelling force between the colloidal particles will be, and consequently the better the dispersion of the colloidal particles will be. When the colloidal particles have a zeta potential>30, this means that the colloidal particles can be stored for long periods of time with low incidence of particle agglutination (A. Saupe et al. (2005), Bio-Medical Materials and Engineering, 15:393-402). Therefore, the zeta potential can be used to determine the stability of colloidal particles.

In this example, nanoparticles of chitosan-marine algal polysaccharides of low degree polymerization which were prepared according to the procedures set forth in Example 11 with 4 minutes of untrasonication and stored at room temperature for different times were subjected to zeta potential analysis. In addition, the variation of the average particle size of nanoparticles of chitosan-marine algal polysaccharides of low degree polymerization according to this invention before and after lyophilization was analyzed. The stability of nanoparticles of chitosan-marine algal polysaccharides of low degree polymerization according to this invention was then determined based on the results of the zeta potential analysis and the average particle size analysis.

### A. The Influence of Storage Time Upon the Zeta Potential of Nanoparticles of Chitosan-Marine Algal Polysaccharides of Low Degree Polymerization

### Experimental Procedures:

The nanoparticles of chitosan-marine algal polysaccharides of low degree polymerization as prepared in Example 13 and stored at room temperature was subjected to zeta potential analysis using a Zeta Potential Analyzer (Zetasizer 3000HS, Malvern Instruments Ltd., U.K.).

### Results:

As can be seen from FIG. 14, nanoparticles of chitosan-marine algal polysaccharides of low degree polymerization were subjected to average particle size analysis immediately after preparation, and the detected average particle size was 95 nm. The average particle size increased to 108 nm after one day of storage, and became 108.3 nm on Day 5, 109.7 nm on Day 10, 110 nm on Day 20, and 110 nm on Day 30. Meanwhile, the nanoparticles of chitosan-marine algal polysaccharides of low degree polymerization were subjected to zeta potential analysis immediately after preparation, and the detected zeta potential was 71.6 mV. The detected zeta potential was 72.4 mV after one day of storage, and became 72.2 mV on Day 10, and 71.2 mV on Day 30. The obtained results reveal that the zeta potential of the nanoparticles of chitosan-marine algal polysaccharides of low degree polymerization according to this invention is very stable and almost unchanged. It is thus concluded that the nanoparticles of chitosan-marine algal polysaccharides of low degree polymerization according to this invention has a high degree of stability with low incidence of aggregation with time of storage.

### B. The Variation of Particle Size of Nanoparticles of Chitosan-Marine Algal Polysaccharides of Low Degree Polymerization Before and After Lyophilization

### Experimental Procedures:

In this experiment, nanoparticles of chitosan-marine algal polysaccharides of low degree polymerization which were prepared according to the procedures set forth in Example 11 with 4 minutes of untrasonication and stored at room temperature for a time were examined by a scanning electron microscope to observe the variation of average particle size thereof before and after lyophilization.

### Results:

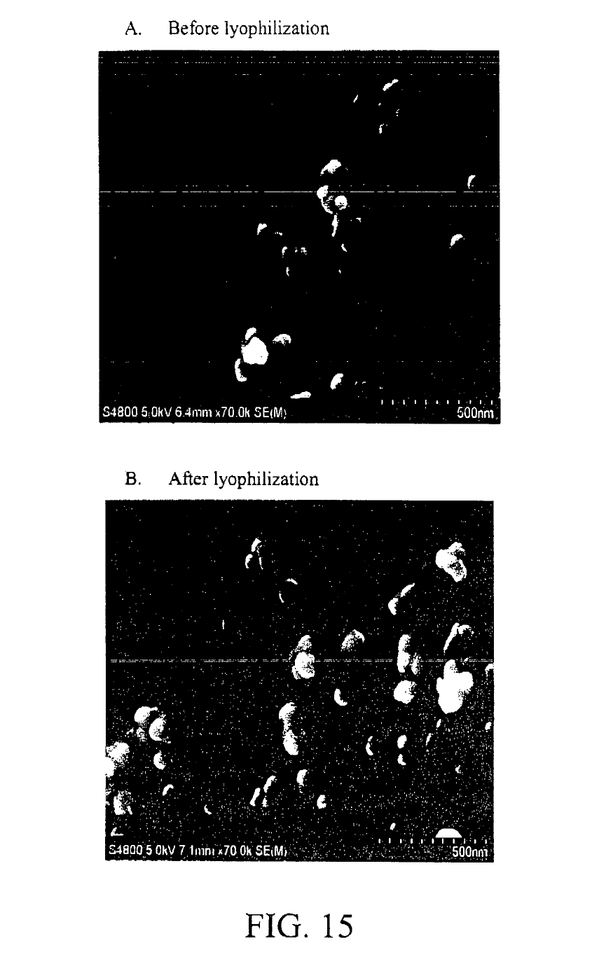

FIG. 15 shows the particle morphology of nanoparticles of chitosan-marine algal polysaccharides of low degree polymerization as observed by scanning electron microscopy (SEM). As can be seen from panels (A) and (B) of FIG. 15, the nanoparticles before and after lyophilization have a spherical shape with an average particle size around 100 nm and no significant change of particle morphology occurred. It is thus concluded that the nanoparticles of chitosan-marine algal polysaccharides of low degree polymerization according to this invention have excellent stability.

### Example 15

### The Influence of the Ultrasonication Time Upon the Zeta Potential of Nanoparticles of Chitosan-Marine Algal Polysaccharides of Low Degree Polymerization

In order to understand the influence of the ultrasonication time upon the zeta potential of nanoparticles of chitosan-marine algal polysaccharides of low degree polymerization, in this Example, nanoparticles of chitosan-marine algal polysaccharides of low degree polymerization prepared with different times of ultrasonication were subjected to zeta potential analysis as described above.

### Experimental Procedures:

Nanoparticles of chitosan-marine algal polysaccharides of low degree polymerization (abbreviated as LDPGP) as prepared according to the procedures set forth in section A of Example 13 were subjected to zeta potential analysis following the procedures described in Example 14.

### Results:

Table 5 shows the zeta potential values of nanoparticles of chitosan-LDPGP prepared with different times of ultrasonication. It can be appreciated from Table 5 that in an acidic chitosan solution having a pH value of 4.5, the amino groups of chitosan were present in the form of positively charged NH3⁺ group (P. A. Sandford (1989), “Chitosan: commercial uses and potential applications” in Chitin and Chitosan: Sources, Chemistry, Biochemistry, Physical Properties and Applications. Skjak-Braek. S. Anthonsen T., and Sandford P. (Ed). Elsevier Applied Science, N.Y.) and, therefore, it was detected to have a positive value of zeta potential. On the other hand, the marine algal polysaccharides of low degree polymerization contained in the marine algal extract according to this invention, on which negatively charged sulfate ions were present, was detected to have a negative value of zeta potential. The results shown in Table 5 reveal that the zeta potential of the nanoparticles of chitosan-LDPGP thus prepared ranges from 30 to 80 mV. It is therefore concluded that nanoparticles of chitosan-marine algal polysaccharides of low degree polymerization according to this invention would have a high degree of particle stability in a colloidal dispersion.

TABLE 5
The Zeta potentials of nanoparticles of chitosan-LDPGP prepared
with different times of ultrasonication.

| | Time of | | |
|---|---|---|---|
| Samples | ultrasonication (min) | Zeta potential (mV) | Width |
| LDPGP | | −52.84 ± 1.61 | 6.7 |
| Chitosan | | 93.69 ± 1.05 | 6.7 |
| Nanoparticles of | 1 | 69.63 ± 4.70 | 6.7 |
| chitosan-LDPGP | | | |
| | 2 | 32.36 ± 4.17 | 6.7 |
| | 3 | 74.85 ± 2.86 | 6.7 |
| | 4 | 71.33 ± 1.97 | 6.7 |
| | 5 | 51.07 ± 2.37 | 6.7 |

Note:
Width refers to the reliability of the detected zeta potential, in which the lower the detected value of width is, the narrower the detected value of particle distribution will be and the higher the reliability will be. Width <10 means that most of the particles are distributed within this area.

### Example 16

### The Effect of Nanoparticles of Chitosan-Marine Algal Polysaccharides of Low Degree Polymerization Upon the Cell Proliferation of Fibroblast Cells

In order to find out whether or not the nanoparticles of chitosan-marine algal polysaccharides of low degree polymerization according to this invention are capable of promoting cell proliferation of fibroblast cells, in this example, various concentrations (0.375˜200 μg/mL) of the nanoparticles which were prepared according to the procedures set forth in Example 11 with 4 minutes of untrasonication and lyophilized were immediately analyzed according to the procedures set forth in Example 8.

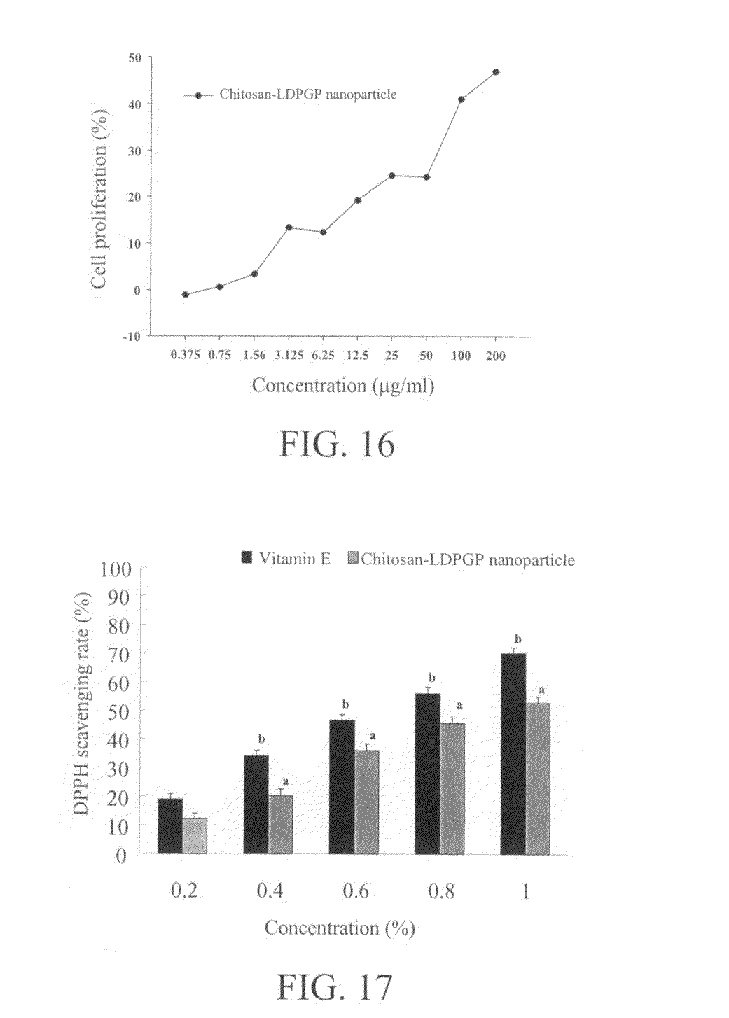

As can be seen from FIG. 16, within the used concentration, the higher the concentration of nanoparticles used, the higher the cell proliferation rate of the fibroblast cells will be. When the nanoparticles of chitosan-marine algal polysaccharides of low degree polymerization according to this invention were used at a concentration of 200 μg/mL, the cell proliferation rate of the fibroblast cells reached almost 50% and tended to increase with an increase of the concentration of nanoparticles. When the cell number of the fibroblast cells is increased, the total amount of collagen produced thereby will likewise increase, thus reaching the effect of enhancing skin elasticity.

### Example 17

### The Effect of Nanoparticles of Chitosan-Marine Algal Polysaccharides of Low Degree Polymerization in Scavenging DPPH Radicals

### Experimental Procedures:

In order to find out whether or not the nanoparticles of chitosan-marine algal polysaccharides of low degree polymerization according to this invention are capable of scavenging DPPH radicals, in this example, various concentrations (0.2, 0.4, 0.6, 0.81% by weight) of the nanoparticles which were prepared according to the procedures set forth in Example 11 with 4 minutes of untrasonication and lyophilized were immediately analyzed substantially according to the procedures set forth in Example 5, with the exception that vitamin E was used as a positive control group.

### Results:

FIG. 17 shows the effect of the nanoparticles of chitosan-marine algal polysaccharides of low degree polymerization according to this invention in scavenging DPPH radicals, as compared to that of the control group (vitamin E). The obtained results reveal that the DPPH radical scavenging activity of the nanoparticles of chitosan-marine algal polysaccharides of low degree polymerization according to this invention tends to increase with an increase of the concentration of the nanoparticles. The higher the scavenging activity is, the higher the ability in inhibiting free radical production will be. It is therefore concluded that the nanoparticles of chitosan-marine algal polysaccharides of low degree polymerization according to this invention are effective in scavenging DPPH radicals.

### Example 18

### The Effect of Nanoparticles of Chitosan-Marine Algal Polysaccharides of Low Degree Polymerization in Scavenging Superoxide Radicals

### Experimental Procedures:

In order to find out whether or not the nanoparticles of chitosan-marine algal polysaccharides of low degree polymerization according to this invention are capable of scavenging superoxide radicals, in this example, various concentrations (0.2, 0.4, 0.6, 0.8, 1% by weight) of the nanoparticles which were prepared according to the procedures set forth in Example 11 with 4 minutes of untrasonication and lyophilized were immediately analyzed substantially according to the procedures set forth in Example 6, the control group used being vitamin C.

### Results:

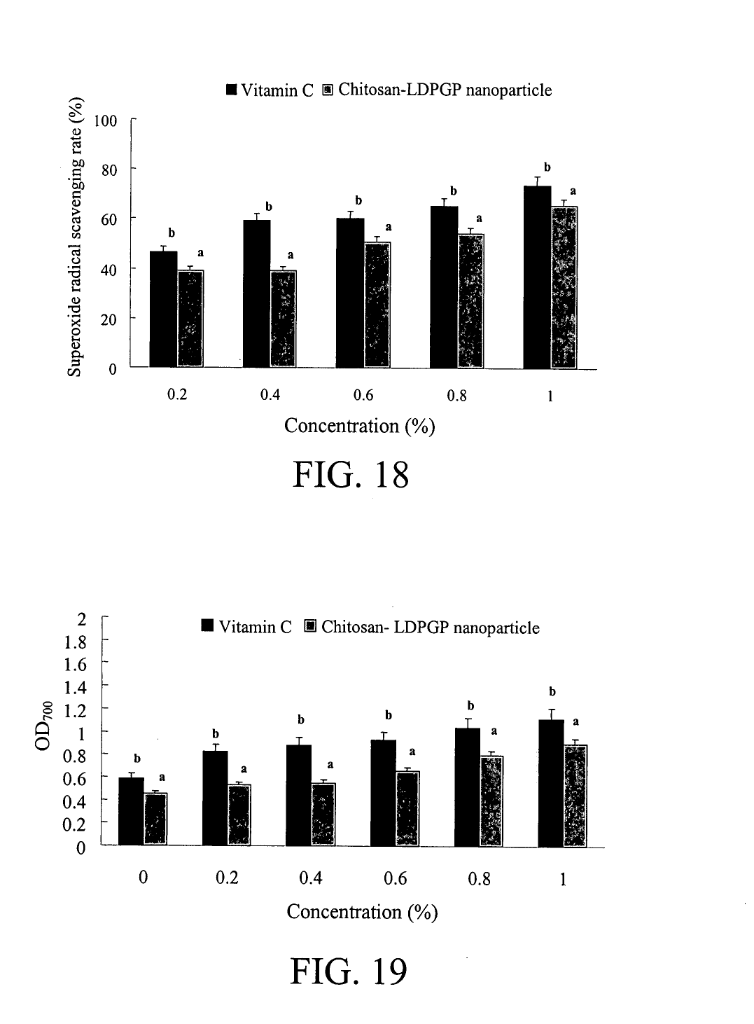

FIG. 18 shows the effect of the nanoparticles of chitosan-marine algal polysaccharides of low degree polymerization according to this invention in scavenging superoxide radicals, as compared to that of the control group (vitamin C). The obtained results reveal that the superoxide radical scavenging activity of the nanoparticles of chitosan-marine algal polysaccharides of low degree polymerization according to this invention tends to slightly increase with an increase of the concentration of the nanoparticles. When the concentration of nanoparticles of chitosan-marine algal polysaccharides of low degree polymerization according to this invention reached 1.0% by weight, the scavenging rate is approaching almost 50% and tends to increase with an increase of the concentration of the nanoparticles. The higher the scavenging activity is, the higher the ability in inhibiting free radical production will be. It is therefore concluded that the nanoparticles of chitosan-marine algal polysaccharides of low degree polymerization according to this invention are effective in scavenging superoxide radicals.

### Example 19

### Detection of the Reducing Power of Nanoparticles of Chitosan-Marine Algal Polysaccharides of Low Degree Polymerization

### Experimental Procedures:

In order to find out whether or not the nanoparticles of chitosan-marine algal polysaccharides of low degree polymerization according to this invention have a reducing power, various concentrations (0.2, 0.4, 0.6, 0.8, 1% by weight) of the nanoparticles which were prepared according to the procedures set forth in Example 11 with 4 minutes of untrasonication and lyophilized were immediately analyzed substantially according to the procedures set forth in Example 7, the control group used being vitamin C.

### Results:

As can be seen from FIG. 19, the detected absorbance increases with an increase of the concentration of the nanoparticles of chitosan-marine algal polysaccharides of low degree polymerization according to this invention. The obtained results indicate that the reducing power of the nanoparticles tends to increase with an increase of the concentration thereof. In addition, the nanoparticles of chitosan-marine algal polysaccharides of low degree polymerization according to this invention exhibit best results at the concentrations of 0.8 and 1% by weight. It is therefore concluded that the nanoparticles of chitosan-marine algal polysaccharides of low degree polymerization according to this invention have excellent reducing power.

### Example 20

### Usage of Nanoparticles of Chitosan-Marine Algal Polysaccharides of Low Degree Polymerization in Skin Care Cosmetic Product

### Experimental Procedures:

In order to explore the usage of the nanoparticles of chitosan-marine algal polysaccharides of low degree polymerization according to this invention in skin care cosmetic products, in this example, a vital cream having the basic formulation as shown in Table 6 was prepared using various concentrations (0.2, 0.4, 0.6, 0.8, 1% by weight) of the nanoparticles which were prepared according to the procedures set forth in Example 11 with 4 minutes of untrasonication and lyophilized were immediately. The vital cream was subjected to skin elasticity analysis according to the procedures set forth in Example 10.

TABLE 6
The basic formulation of a vital cream containing the nanoparticles
of chitosan-marine algal polysaccharides of low degree
polymerization according to this invention

| Phases | Ingredients | Used amounts (g) |
|---|---|---|
| A (aqueous phase) | Water | 76.35 |
| | KOH | 0.2 |
| | Propylene glycol | 5.0 |
| | Methyl paraben (M.P) | 0.1 |
| | the nanoparticles | 0.25 |
| B (oil phase) | Stearic acid | 5.0 |
| | Cetyl alcohol | 4.0 |
| | Wickenol 158 | 6.0 |
| | P.P. | 0.1 |
| | GMS 1330 surfactant | 1 |

### Experimental Procedures:

The vital cream which has the basic formulation as shown in Table 6 was prepared as follows: A portion of water was substituted by an aqueous solution of nanoparticles of chitosan-marine algal polysaccharides of low degree polymerization as prepared according to the procedures set forth in Example 11. The phase A and the phase B were separately placed into a 70° C. water bath. After being completely dissolved by heating, the two phases were allowed to stand at that temperature for a further 20 minutes, and then removed from the water bath. The phase B was slowly added into the phase A to cause emulsification, followed by homogenization with a homogenizer (PT-MR 3000, Polytron). After cooling, a vital cream containing the nanoparticles of chitosan-marine algal polysaccharides of low degree polymerization according to this invention was obtained. A cream of the same formulation but not containing the nanoparticles was used as a control group.

The vital cream thus prepared was subjected to skin elasticity analysis according to the procedures set forth in Example 10.

### Results:

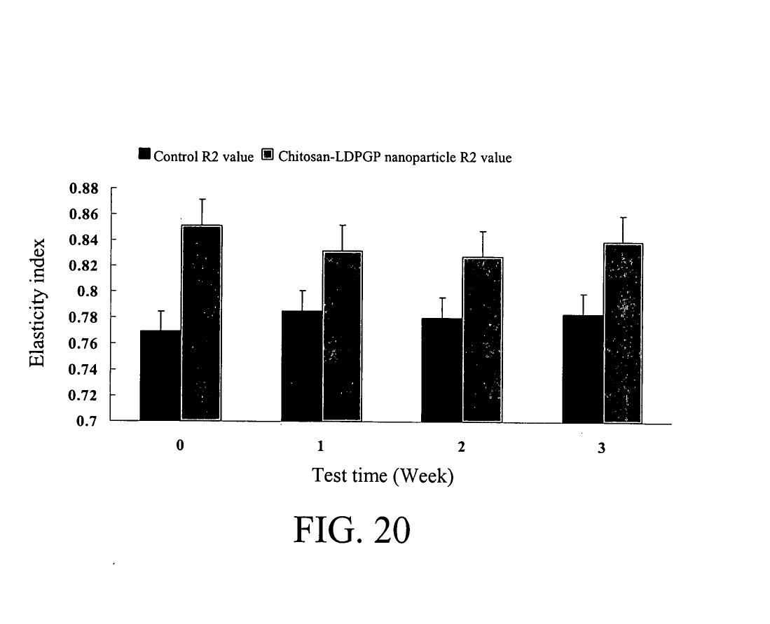

FIG. 20 shows the variation of skin elasticity caused by the application of a vital cream containing the nanoparticles of chitosan-marine algal polysaccharides of low degree polymerization according to this invention or a control cream to the marked areas located on the inner side of each volunteer's lower arm for three weeks. The results reveal that the R2 value significantly increases after a continuous application of the vital cream for 1 week, and continues to increases after 2 and 3 weeks of application. It is thus concluded that the vital cream containing the nanoparticles of chitosan-marine algal polysaccharides of low degree polymerization according to this invention is effective in improving skin elasticity and, hence, is a promising agent for anti-aging of skin.

All patents and literature references cited in the present specification as well as the references described therein, are hereby incorporated by reference in their entirety. In case of conflict, the present description, including definitions, will prevail.

While the invention has been described in connection with specific embodiments thereof, it will be understood that it is capable of further modifications and this application is intended to cover any variations, uses, or adaptations of the invention following, in general, the principles of the invention and including such departures from the present customary practice within the art to which the invention pertains and as may be applied to the essential features hereinbefore set forth, and as follows in the scope of the appended claims.

We claim:

1. A marine algal extract produced by a process comprising the steps of:
(a) extracting a marine algal material with water at an elevated temperature, followed by removal of water insoluble substances, so that an aqueous extract containing marine algal polysaccharides is obtained;
(b) admixing the aqueous extract obtained from step (a) with an acid or an aqueous solution containing said acid so as to form an acidic aqueous solution;
(c) subjecting the acidic aqueous solution thus formed from step (b) to a refining treatment selected from heating treatments and ultrasonication treatments, so that a product containing acid-hydrolyzed marine algal polysaccharides is obtained; and
(d) subjecting the product obtained from step (c) to a ultrafiltration treatment having a molecular weight cut-off value ranging from 1×10² to 5×10⁴ Daltons, so that a marine algal extract comprising marine algal polysaccharides of low degree polymerization is obtained.

2. The marine algal extract as claimed in claim 1, wherein in step (a) of said process, the marine algal material used belongs to any of the following: a marine alga of Gracilaria genus, and a marine alga of the family Gelidiaceae.

3. The marine algal extract as claimed in claim 1, wherein in step (a) of said process, the marine algal material used belongs to any of the following: Gracilaria coforvoides, Gracilaria gigas, Gracilaria chorda, Gracilaria lichenoides, Gracilaria compressa, Gracilaria arcuata, Gracilaria blodgettii, Gracilaria bursa-pastoris, Gracilaria canaliculata, Gracilaria lemaneformis, Gracilaria coronopifolia, Gracilaria edulis, Gracilaria eucheumoides, Gracilaria gracilis, Gracilaria incurvata, Gracilaria punctata, Gracilaria salicomia, Gracilaria spinulosa, Gracilaria srilankia, Gracilaria textori, Gracilaria veillardii, Gelidium amansii, Gelidium corneum, Gelidium crinale, Gelidium divaricatum, Gelidium elegans, Gelidium foliaceum, Gelidium japonicum, Gelidium kintaroi, Gelidium latiusculum, Gelidium pacificum, Gelidium planiusculum, Gelidium pusillim, Gelidium pusillum, Gelidium yamadae, Pterocladia tenuis, Pterocladia nana, and Pterocladiella capillacea.

4. The marine algal extract as claimed in claim 1, wherein in said process, step (a) is conducted at a temperature ranging from 70° C. to 100° C. for a period of from 1 to 6 hours.

5. The marine algal extract as claimed in claim 1, wherein in step (a) of said process, removal of water insoluble substances is conducted by filtration or centrifugation.

6. The marine algal extract as claimed in claim 1, wherein in said process, the aqueous extract obtained from step (a) is in the form of an aqueous solution and is admixed with said acid in step (b).

7. The marine algal extract as claimed in claim 1, wherein in said process, the aqueous extract obtained from step (a) is in the form of a lyophilized powder and is admixed with said aqueous solution containing said acid in step (b).

8. The marine algal extract as claimed in claim 1, wherein in step (b) of said process, said acid is an organic acid selected from the group consisting of acetic acid, formic acid, lactic acid, malic acid, oxalic acid, citric acid, and combinations thereof.

9. The marine algal extract as claimed in claim 1, wherein in step (b) of said process, said acid is an inorganic acid selected from the group consisting of hydrochloric acid, nitric acid, phosphoric acid, and combinations thereof.

10. The marine algal extract as claimed in claim 1, wherein in step (b) of said process, said acid has a concentration in the range of from 0.01% to 30%.

11. The marine algal extract as claimed in claim 1, wherein in step (b) of said process, said acid or said aqueous solution containing said acid is an aqueous acetic acid solution having a concentration in the range of from 0.01% to 30%.

12. The marine algal extract as claimed in claim 1, wherein in step (c) of said process, the acidic aqueous solution thus formed from step (b) is subjected to a heating treatment.

13. The marine algal extract as claimed in claim 12, wherein the heating treatment is conducted at a temperature ranging from 70° C. to 100° C.

14. The marine algal extract as claimed in claim 12, wherein the heating treatment is conducted for a period of from 0.1 to 10 hours.

15. The marine algal extract as claimed in claim 1, wherein in step (c) of said process, the acidic aqueous solution thus formed from step (b) is subjected to a ultrasonication treatment.

16. The marine algal extract as claimed in claim 15, wherein the ultrasonication treatment is conducted at a temperature ranging from 70° C. to 100° C.

17. The marine algal extract as claimed in claim 15, wherein the ultrasonication treatment is conducted at a power of from 10 to 1,000 watts.

18. The marine algal extract as claimed in claim 1, comprising marine algal polysaccharides of low degree polymerization that have a molecular weight in the range of from 1×10² to 1×10⁴ Daltons.

19. The marine algal extract as claimed in claim 18, comprising marine algal polysaccharides of low degree polymerization that have a molecular weight in the range of from 1×10² to 5×10³ Daltons.

20. A pharmaceutical composition comprising a marine algal extract as claimed in claim 1.

21. A method for inhibiting the growth of tumor cells in a subject, comprising administering to the subject a marine algal extract as claimed in claim 1.

22. The method as claimed in claim 21, wherein the tumor cells are melanoma cells.

23. A method for promoting fibroblast proliferation and/or collagen synthesis in a subject, comprising administering to the subject a marine algal extract as claimed in claim 1.

24. A method for promoting wound healing in a subject, comprising administering to the subject a marine algal extract as claimed in claim 1.

25. A cosmetic product comprising a marine algal extract as claimed in claim 1.

26. A process for producing a marine algal extract, comprising the steps of:
(a) extracting a marine algal material with water at an elevated temperature, followed by removal of water insoluble substances, so that an aqueous extract containing marine algal polysaccharides is obtained;
(b) admixing the aqueous extract obtained from step (a) with an acid or an aqueous solution containing said acid so as to form an acidic aqueous solution;
(c) subjecting the acidic aqueous solution thus formed from step (b) to a refining treatment selected from heating treatments and ultrasonication treatments, so that a product containing acid-hydrolyzed marine algal polysaccharides is obtained; and
(d) subjecting the product obtained from step (c) to a ultrafiltration treatment having a molecular weight cut-off value ranging from 1×10² to 5×10⁴ Daltons, so that a marine algal extract comprising marine algal polysaccharides of low degree polymerization is obtained.

27. The process as claimed in claim 26, wherein the marine algal material used in step (a) belongs to any of the following: a marine alga of Gracilaria genus, and a marine alga of the family Gelidiaceae.

28. The process as claimed in claim 26, wherein the marine algal material used in step (a) belongs to any of the following: Gracilaria coforvoides, Gracilaria gigas, Gracilaria chorda, Gracilaria lichenoides, Gracilaria compressa, Gracilaria arcuata, Gracilaria blodgettii, Gracilaria bursa-pastoris, Gracilaria canaliculata, Gracilaria lemaneformis, Gracilaria coronopifolia, Gracilaria edulis, Gracilaria eucheumoides, Gracilaria gracilis, Gracilaria incurvata, Gracilaria punctata, Gracilaria salicornia, Gracilaria spinulosa, Gracilaria srilankia, Gracilaria textorii, Gracilaria veillardii, Gelidium amansii, Gelidium corneum, Gelidium crinale, Gelidium divaricatum, Gelidium elegans, Gelidium foliaceum, Gelidium japonicum, Gelidium kintaroi, Gelidium latiusculum, Gelidium pacificum, Gelidium planiusculum, Gelidium pusillim, Gelidium pusillum, Gelidium yamadae, Pterocladia tenuis, Pterocladia nana, and Pterocladiella capillacea.

29. The process as claimed in claim 26, wherein step (a) is conducted at a temperature ranging from 70° C. to 100° C. for a period of from 1 to 6 hours.

30. The process as claimed in claim 26, wherein in step (a), removal of water insoluble substances is conducted by filtration or centrifugation.

31. The process as claimed in claim 26, wherein the aqueous extract obtained from step (a) is in the form of an aqueous solution and is admixed with said acid in step (b).

32. The process as claimed in claim 26, wherein the aqueous extract obtained from step (a) is in the form of a lyophilized powder and is admixed with said aqueous solution containing said acid in step (b).

33. The process as claimed in claim 26, wherein said acid used in step (b) is an organic acid selected from the group consisting of acetic acid, formic acid, lactic acid, malic acid, oxalic acid, citric acid, and combinations thereof.

34. The process as claimed in claim 26, wherein said acid used in step (b) is an inorganic acid selected from the group consisting of hydrochloric acid, nitric acid, phosphoric acid, and combinations thereof.

35. The process as claimed in claim 26, wherein said acid used in step (b) has a concentration in the range of from 0.01% to 30%.

36. The process as claimed in claim 26, wherein said acid or said aqueous solution containing said acid used in step (b) is an aqueous acetic acid solution having a concentration in the range of from 0.01% to 30%.

37. The process as claimed in claim 26, wherein in step (c), the acidic aqueous solution thus formed from step (b) is subjected to a heating treatment.

38. The process as claimed in claim 37, wherein the heating treatment is conducted at a temperature ranging from 70° C. to 100° C.

39. The process as claimed in claim 37, wherein the heating treatment is conducted for a period of from 0.1 to 10 hours.

40. The process as claimed in claim 26, wherein in step (c), the acidic aqueous solution thus formed from step (b) is subjected to a ultrasonication treatment.

41. The process as claimed in claim 40, wherein the ultrasonication treatment is conducted at a temperature ranging from 70° C. to 100° C.

42. The process as claimed in claim 40, wherein the ultrasonication treatment is conducted at a power of from 10 to 1,000 watts.

43. The process as claimed in claim 26, wherein the marine algal extract thus obtained from the process comprises marine algal polysaccharides of low degree polymerization that have a molecular weight in the range of from 1×10² to 1×10⁴ Daltons.

44. The process as claimed in claim 43, wherein the marine algal extract thus obtained from the process comprises marine algal polysaccharides of low degree polymerization that have a molecular weight in the range of from 1×10² to 5×10³ Daltons.

45. A nanoparticle of chitosan-marine algal polysaccharides of low degree polymerization, said nanoparticle being produced by a process comprising the steps of:
(a) providing a reaction mixture by admixing a first aqueous solution containing chitosan and an acid with a second aqueous solution containing a marine algal extract as claimed in claim 1; and
(b) subjecting the reaction mixture to a ultrasonication treatment, so that a third aqueous solution containing the nanoparticle is obtained.

46. The nanoparticle as claimed in claim 45, wherein in step (a) of said process, the used amount of the first aqueous solution versus that of the second aqueous solution is within the range of from 1:1 to 10:1.

47. The nanoparticle as claimed in claim 45, wherein in step (a) of said process, the first aqueous solution has a concentration of chitosan in the range of from 0.002% to 1.0%.

48. The nanoparticle as claimed in claim 45, wherein in step (a) of said process, the second aqueous solution has a concentration of marine algal polysaccharides of low degree polymerization in the range of from 0.001% to 0.5%.

49. The nanoparticle as claimed in claim 45, wherein in step (a) of said process, the acid contained in the first aqueous solution is an organic acid selected from the group consisting of acetic acid, formic acid, lactic acid, malic acid, oxalic acid, citric acid, and combinations thereof.

50. The nanoparticle as claimed in claim 45, wherein in step (a) of said process, the acid contained in the first aqueous solution is an inorganic acid selected from the group consisting of hydrochloric acid, nitric acid, phosphoric acid, and combinations thereof.

51. The nanoparticle as claimed in claim 45, wherein in step (a) of said process, the used first aqueous solution comprises an aqueous acetic acid solution having a concentration in the range of from 0.01% to 30%.

52. The nanoparticle as claimed in claim 45, wherein in step (b) of said process, the ultrasonication treatment is conducted at a temperature ranging from 4° C. to 50° C.

53. The nanoparticle as claimed in claim 45, wherein in step (b) of said process, the ultrasonication treatment is conducted at a power of from 20 to 100 watts.

54. The nanoparticle as claimed in claim 45, wherein in step (b) of said process, the ultrasonication treatment is conducted for a period of from 1 to 60 minutes.

55. The nanoparticle as claimed in claim 45, wherein the third aqueous solution thus obtained from step (b) is further purified by the following step:
(c) subjecting the third aqueous solution thus obtained from step (b) to a high-speed centrifugation treatment, so that a supernatant containing the nanoparticle may be collected.

56. The nanoparticle as claimed in claim 55, wherein the high-speed centrifugation treatment is conducted at a speed ranging from 5,000 to 20,000 rpm.

57. A pharmaceutical composition comprising a nanoparticle of chitosan-marine algal polysaccharides of low degree polymerization as claimed in claim 45.

58. A method for promoting fibroblast proliferation in a subject, comprising administering to the subject a nanoparticle of chitosan-marine algal polysaccharides of low degree polymerization as claimed in claim 45.

59. A method for promoting wound healing in a subject, comprising administering to the subject a nanoparticle of chitosan-marine algal polysaccharides of low degree polymerization as claimed in claim 45.

60. A cosmetic product comprising a nanoparticle of chitosan-marine algal polysaccharides of low degree polymerization as claimed in claim 45.

61. A process for producing a nanoparticle of chitosan-marine algal polysaccharides of low degree polymerization, said process comprising the steps of:
(a) providing a reaction mixture by admixing a first aqueous solution containing chitosan and an acid with a second aqueous solution containing a marine algal extract as claimed in claim 1; and
(b) subjecting the reaction mixture to a ultrasonication treatment, so that a third aqueous solution containing the nanoparticle is obtained.

62. The process as claimed in claim 61, wherein in step (a), the used amount of the first aqueous solution versus that of the second aqueous solution is within the range of from 1:1 to 10.1.

63. The process as claimed in claim 61, wherein the first aqueous solution used in step (a) has a concentration of chitosan in the range of from 0.002% to 1.0%.

64. The process as claimed in claim 61, wherein the second aqueous solution used in step (a) has a concentration of marine algal polysaccharides of low degree polymerization in the range of from 0.001% to 0.5%.

65. The process as claimed in claim 61, wherein in step (a), the acid contained in the first aqueous solution is an organic acid selected from the group consisting of acetic acid, formic acid, lactic acid, malic acid, oxalic acid, citric acid, and combinations thereof.

66. The process as claimed in claim 61, wherein in step (a), the acid contained in the first aqueous solution is an inorganic acid selected from the group consisting of hydrochloric acid, nitric acid, phosphoric acid, and combinations thereof.

67. The process as claimed in claim 61, wherein the first aqueous solution used in step (a) comprises an aqueous acetic acid solution having a concentration in the range of from 0.01% to 30%.

68. The process as claimed in claim 61, wherein in step (b), the ultrasonication treatment is conducted at a temperature ranging from 4° C. to 50° C.

69. The process as claimed in claim 61, wherein in step (b), the ultrasonication treatment is conducted at a power of from 20 to 100 watts.

70. The process as claimed in claim 61, wherein in step (b), the ultrasonication treatment is conducted for a period of from 1 to 60 minutes.

71. The process as claimed in claim 61, wherein the third aqueous solution thus obtained from step (b) is further purified by the following step:
(c) subjecting the third aqueous solution thus obtained from step (b) to a high-speed centrifugation treatment, so that a supernatant containing the nanoparticle may be collected.

72. The nanoparticle as claimed in claim 71, wherein the high-speed centrifugation treatment is conducted at a speed ranging from 5,000 to 20,000 rpm.
